(19) United States
(12) Patent Application Publication
(10) Pub. No.: US 2014/0100558 A1
(43) Pub. Date: Apr. 10, 2014

(54) MICRO-ARTICULATED SURGICAL INSTRUMENTS USING MICRO GEAR ACTUATION

(71) Applicants: Gregory P. Schmitz, Los Gatos, CA (US); Juan Diego Perea, Campbell, CA (US); Ronald Leguidleguid, Union City, CA (US); Gregory B. Arcenio, Redwood City, CA (US)

(72) Inventors: Gregory P. Schmitz, Los Gatos, CA (US); Juan Diego Perea, Campbell, CA (US); Ronald Leguidleguid, Union City, CA (US); Gregory B. Arcenio, Redwood City, CA (US)

(21) Appl. No.: 13/855,627

(22) Filed: Apr. 2, 2013

Related U.S. Application Data
(60) Provisional application No. 61/710,608, filed on Oct. 5, 2012.

(51) Int. Cl. A61B 17/32; A61B 18/18; A61B 17/34; A61B 17/3201; A61B 17/285

(52) U.S. Cl. CPC A61B 17/32002; A61B 17/3201; A61B 17/285; A61B 17/3403; A61B 18/18
USPC 606/33; 606/1; 606/180; 606/174; 606/205; 606/185

(57) ABSTRACT

A medical device for removing or manipulating tissue of a subject is provided with a distal housing having an end effector, and an elongate member configured to introduce the distal housing to a target tissue site of the subject. The elongate member may have proximal and distal portions interconnected by a joint mechanism that is configured to allow the two portions to articulate relative to one another. In some embodiments, the joint mechanism includes one or more nested crown gear(s) configured to drive associated spur gear(s) to accomplish the articulation. In some embodiments, the end effector is a powered scissors device.

### CROSS REFERENCE TO RELATED APPLICATIONS

This application claims the benefit of Provisional Application No. 61/710,608 filed on Oct. 5, 2012.

This application is related to the following U.S. applications: application Ser. No. 13/843,462 filed Mar. 15, 2013; application Ser. No. 13/535,197 filed Jun. 27, 2012; application Ser. No. 13/388,653 filed Apr. 16, 2012; application Ser. No. 13/289,994 filed Nov. 4, 2011; application Ser. No. 13/007,578 filed Jan. 14, 2011; application Ser. No. 12/491,220 filed Jun. 24, 2009; application Ser. No. 12/490,301 filed Jun. 23, 2009; application Ser. No. 12/490,295 filed Jun. 23, 2009; Provisional Application No. 61/408,558 filed Oct. 29, 2010; Provisional Application No. 61/234,989 filed Aug. 18, 2009; Provisional Application No. 61/075,007 filed Jun. 24, 2008; Provisional Application No. 61/075,006 filed Jun. 23, 2008; Provisional Application No. 61/164,864 filed Mar. 30, 2009; and Provisional Application No. 61/164,883 filed Mar. 30, 2009.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the present disclosure relate to micro-scale and millimeter-scale tissue debridement devices that may, for example, be used to remove unwanted tissue or other material from selected locations within a body of a patient during a minimally invasive or other medical procedure, and in particular embodiments, multi-layer, multi-material electrochemical fabrication methods that are used to, in whole or in part, form such devices.

### BACKGROUND

Debridement is the medical removal of necrotic, cancerous, damaged, infected or otherwise unwanted tissue. Some medical procedures include, or consist primarily of, the mechanical debridement of tissue from a subject. Rotary debrider devices have been used in such procedures for many years.

Some debrider devices with relatively large dimensions risk removing unintended tissue from the subject, or damaging the unintended tissue. There is a need for tissue removal devices which have small dimensions and improved functionality which allow them to more safely remove only the desired tissue from the patient. There is also a need for tissue removal devices which have small dimensions and improved functionality over existing products and procedures which allow them to more efficiently remove tissue from the patient.

Prior art tissue removal devices often remove tissue in large pieces, having dimensions well over 2 mm. The tissue pieces are removed through an aspiration lumen typically 3.5 to 5 mm in diameter. Since the tissue pieces being removed commonly have dimensions that are 1 to 2 lumen diameters in length, the tissue pieces can often clog the tissue removal lumen.

One portion of the body in which tissue can be removed to treat a variety of conditions is the spine area. Tissue removal devices for the spine are needed that can be produced with sufficiently small dimensions and/or that have increased performance over existing techniques. For example, a herniated disc or bulging disc can be treated by performing a discectomy, e.g. by removing all or part of the nucleus pulposus of the damaged disc. Such procedures may also involve a laminotomy or laminectomy wherein a portion or all of a lamina may be removed to allow access to the herniated disc. Artificial disc replacement (total or partial) is another example of a procedure which requires the removal of all or a portion of the disc, which is replaced with an artificial device or material.

Tissue removal devices are needed which can be produced with sufficient mechanical complexity and a small size so that they can both safely and more efficiently remove tissue from a subject, and/or remove tissue in a less invasive procedure and/or with less damage to adjacent tissue such that risks are lowered and recovery time is improved.

### SUMMARY OF THE DISCLOSURE

According to some aspects of the disclosure, a medical device for manipulating tissue of a subject is provided. One exemplary device includes a distal housing, an elongate member, a joint mechanism, proximal and distal crown gears and a spur gear. In this exemplary embodiment, the distal housing is configured with an end effector. The elongate member is coupled to the distal housing and is configured to introduce the distal housing to a target tissue site of the subject. The elongate member comprises a proximal portion having a first central axis and a distal portion having a second central axis. The proximal portion of the elongate member comprises a proximal outer tube and a proximal inner drive tube rotatably mounted within the proximal outer tube. The distal portion of the elongate member comprises a distal outer tube and a distal inner drive tube rotatably mounted within the distal outer tube. The distal inner drive tube engages with a portion of the end effector to drive the end effector. The joint mechanism is configured to pivotably connect a distal end of the proximal outer tube with a proximal end of the distal outer tube. The joint mechanism allows the distal portion of the elongate member to be pivoted relative to the proximal portion such that an angle formed between the first and the second central axes can be changed. The proximal crown gear is located at a distal end of the proximal inner drive tube. The distal crown gear is located at a proximal end of the distal inner drive tube. The spur gear spans between and inter-engages with the proximal crown gear and the distal crown gear, thereby allowing the end effector to be positioned by the proximal and the distal outer tubes, and to be driven by the proximal inner drive tube, the spur gear and the distal inner drive tube.

In some embodiments, the end effector comprises a rotary tissue cutter assembly. The rotary tissue cutter assembly may comprise at least one rotatable member that rotates about the second central axis, or that has an axis of rotation that is perpendicular to the second central axis. In some embodiments, the distal inner drive tube comprises a first lumen and the proximal inner drive tube comprises a second lumen. In these embodiments, the first lumen is in fluid communication with the tissue cutter assembly and the second lumen is in fluid communication with the first lumen through the joint mechanism. The tissue cutter assembly, the first lumen, the joint mechanism and the second lumen may be configured to cooperate to transport tissue debris cut by the tissue cutter assembly in a proximal direction through the first lumen, the joint mechanism and the second lumen.

In some embodiments, the end effector may include a pair of scissor blades configured to cut tissue, a pair of tissue grasper jaws and/or a needle driver.

In some embodiments, the proximal portion of the elongate member further includes a proximal inner articulation tube rotatably mounted within the proximal outer tube. In these embodiments, the proximal inner articulation tube includes a crown gear on a distal end thereof configured to mesh with a gear segment of the joint mechanism to pivotably drive the distal portion of the elongate member relative to the proximal portion of the elongate member.

In some embodiments, the proximal portion of the elongate member includes a second proximal inner drive tube rotatably mounted within the proximal outer tube. In these embodiments the distal portion of the elongate member includes a second distal inner drive tube rotatably mounted within the distal outer tube. The second distal inner drive tube is configured to engage with a portion of the end effector to drive the end effector. The device further includes a second proximal crown gear located at a distal end of the second proximal inner drive tube, a second distal crown gear located at a proximal end of the second distal inner drive tube, and a second spur gear spanning between and inter-engaging with the second proximal crown gear and the second distal crown gear.

In some embodiments, the end effector includes a pair of tissue grasper jaws. One of the pair of tissue grasper jaws may be configured to be rotatably driven by a crown gear located on a distal end of the first distal inner drive tube. The other of the pair of tissue grasper jaws may be configured to be rotatably driven by a crown gear located on a distal end of the second distal inner drive tube. With this arrangement, each of the pair of tissue grasper jaws may be independently rotated relative to the second central axis and may be rotated between an open jaw position and a closed jaw position.

In some embodiments, the proximal portion of the elongate member includes a second proximal drive tube rotatably mounted coaxially with the proximal outer tube. In these embodiments, the distal portion of the elongate member includes a second distal drive tube rotatably mounted coaxially with the distal outer tube. The second distal drive tube engages with a portion of the end effector to support the end effector. The device may further include a second proximal crown gear located at a distal end of the second proximal drive tube, a second distal crown gear located at a proximal end of the second distal drive tube, and a second spur gear spanning between and inter-engaging with the second proximal crown gear and the second distal crown gear. This arrangement permits the rotational orientation of the end effector relative to the second central axis to be changed by rotating the second distal drive tube with the second proximal drive tube and second spur gear. The proximal and the distal portions of the elongate member may be configured to rotate together about the first central axis relative to a more proximal portion of the device.

In some embodiments, the device may include a second spur gear spanning between and inter-engaging with the proximal crown gear and the distal crown gear, thereby allowing the end effector to be driven by the proximal inner drive tube, the first and second spur gears and the distal inner drive tube. In these embodiments, the first and the second spur gears provide a dual load path between the proximal and the distal inner drive tubes.

According to aspects of the disclosure, methods of manipulating tissue of a subject are provided. In some embodiments, the method includes providing a device having a distal housing configured with an end effector and an elongate member coupled to the distal housing. The method may further include introducing the distal housing to a target tissue site of the subject with the elongate member. The end effector may be driven with a drive train comprising a proximal crown gear located at a distal end of a proximal drive tube, a distal crown gear located at a proximal end of a distal drive tube, and a first spur gear spanning between and inter-engaging with the proximal crown gear and the distal crown gear. The method may further include pivoting the location of the end effector, the distal housing and the distal drive tube relative to the proximal drive tube by rotating a second proximal tube. The second proximal tube is rotatably mounted coaxially with the proximal drive tube in these embodiments and has a crown gear located on a distal end. The crown gear engages with a gear segment coaxially mounted with the spur gear. The methods further include manipulating the tissue of the subject with the end effector.

In some of the above embodiments, the end effector includes a rotary tissue cutter assembly. The rotary tissue cutter assembly may include at least one rotatable member that rotates about a central axis of the distal drive tube, or has an axis of rotation that is perpendicular to a central axis of the distal drive tube. The end effector may include a pair of scissor blades configured to cut tissue, a pair of tissue grasper jaws and/or a needle driver. The pivoting step in the above embodiments may include a computer receiving movement inputs from a surgeon and providing electrical outputs to drive an electric motor coupled to the second proximal tube.

According to aspects of the disclosure, a powered scissors device is provided. In some embodiments the scissors device includes a distal housing, an elongate member, a rotatably blade, a crown gear and a spur gear. In these embodiments the distal housing has a fixed cutting arm located thereon. The elongate member is coupled to the distal housing and is configured to introduce the distal housing to a target tissue site of the subject. The elongate member includes an outer tube and an inner drive tube rotatably mounted within the outer tube. The rotatable blade is rotatably mounted to the distal housing and has at least one cutting element configured to cooperate with the fixed arm to shear tissue therebetween. The crown gear is located at a distal end of the inner drive tube. The first spur gear is configured to inter-engage with the crown gear and is coupled with the rotatable blade to allow the crown gear to drive the rotatable blade.

In some embodiments, the rotatable blade has an axis of rotation that is perpendicular to an axis of rotation of the inner drive tube. The rotatable blade may be partially located within a slot formed within the distal housing such that the at least one cutting element is covered by the distal housing during at least half of its rotation about an axis of rotation of the rotatable blade.

Other aspects of the disclosure will be understood by those of skill in the art upon review of the teachings herein. Other aspects of the disclosure may involve combinations of the above noted aspects of the disclosure. These other aspects of the disclosure may provide various combinations of the aspects presented above as well as provide other configurations, structures, functional relationships, and processes that have not been specifically set forth above.

### BRIEF DESCRIPTION OF THE DRAWINGS

FIGS. 1-3 illustrate an exemplary embodiment of a working end of a tissue removal device.

FIGS. 4A-4G illustrate exemplary embodiments of drive mechanisms which can power the drive trains in the working end of tissue removal devices.

FIGS. 5A-5C show another exemplary embodiment of a tissue removal device.

FIGS. 6A-6C show an exemplary cutter head assembly 5332 that may be used with debriding device 5310, shown in FIGS. 5A-5C.

FIGS. 7A-7F show details of an exemplary rotor housing assembly 5420′.

FIGS. 8A-8B show a portion of an exemplary embodiment of an articulating tissue cutter.

FIG. 9 shows a crown gear meshing with the spur gear of the articulating tissue cutter of FIGS. 8A-8B.

FIGS. 10A-10B show a portion of another exemplary embodiment of an articulating tissue cutter.

FIGS. 11A-11B show a portion of an exemplary embodiment of surgical scissors.

FIGS. 12A-12C show a portion of an exemplary embodiment of tissue graspers.

FIGS. 13A-13I show a portion of another exemplary embodiment of tissue graspers.

FIGS. 14A-14F show a portion of an exemplary embodiment of an articulating tissue grasper.

FIG. 15 shows a portion of another exemplary embodiment of an articulating tissue grasper.

FIG. 16 shows a portion of an exemplary embodiment of an axially driven linear tool.

FIG. 17 shows a portion of an exemplary embodiment of a radially driven linear tool.

FIG. 18 is a top perspective view showing an exemplary embodiment of a powered scissors device.

FIG. 19 is a bottom perspective view showing the scissors device of FIG. 18.

FIG. 20 is a top plan view showing the scissors device of FIG. 18.

FIG. 21 is a side elevation view showing the scissors device of FIG. 18.

FIG. 22 is a bottom view showing the scissors device of FIG. 18.

FIG. 23 is an exploded view showing the scissors device of FIG. 18.

FIG. 24 is a side elevation view showing the distal housing or lug of the scissors device of FIG. 18.

FIG. 25 is a distal end view showing the distal housing or lug of the scissors device of FIG. 18.

FIG. 26 is a proximal end view showing the distal housing or lug of the scissors device of FIG. 18.

### DETAILED DESCRIPTION

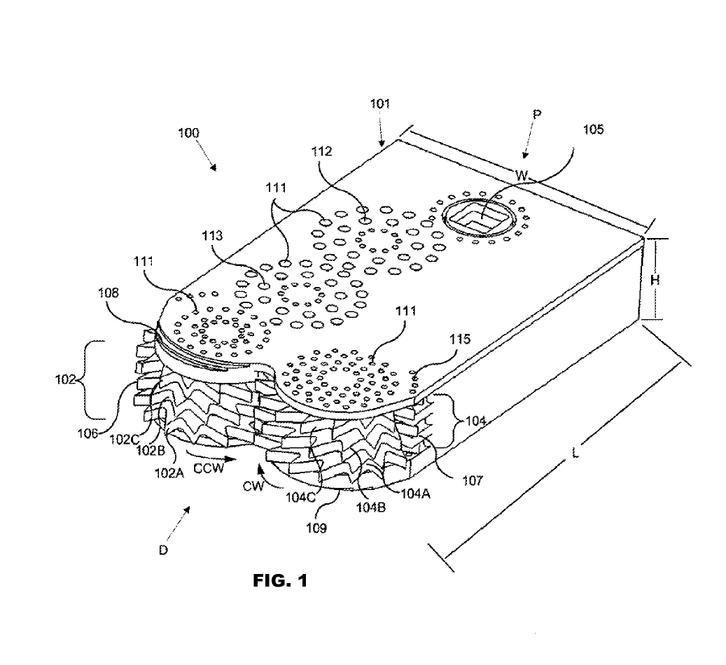

FIGS. 1-3 illustrate an exemplary embodiment of a working end of a tissue removal device, which can be fabricated wholly or in part by electrochemical fabrication techniques, such as those described or referenced herein. Tissue removal device working end 100 has a distal region “D” and proximal region “P,” and includes housing 101 and blade stacks 102 and 104. Blade stacks 102 and 104 include a plurality of blades 102A-102C and 104A-104C, respectively. Three blades are shown in each stack, although the blade stacks can have one or more blades. Each of the blades includes a plurality of teeth 106 (see FIG. 3), some of which are shown projecting from housing 101 and configured to engage and process tissue. Processing tissue as used herein includes any of cutting tissue, shredding tissue, capturing tissue, any other manipulation of tissue as described herein, or any combination thereof. The working end of the device generally has a length L, height H, and width W. Housing 101 can have a variety of shapes or configurations, including a generally cylindrical shape.

In this embodiment both blade stacks are configured to rotate. The blades in blade stack 102 are configured to rotate in a direction opposite that of the blades in blade stack 104, as designated by the counterclockwise “CCW” and clockwise “CW” directions in FIG. 1. The oppositely rotating blades direct material, such as tissue, into an interior region of housing 101 (described in more detail below). In some embodiments, the blades can be made to be rotated in directions opposite to those indicated, e.g. to disengage from tissue if a jam occurs or to cause the device to be pulled distally into a body of tissue when given appropriate back side teeth configurations.

Housing 101 also includes a drive mechanism coupler 105, shown as a square hole or bore, which couples a drive train disposed in the housing to a drive mechanism disposed external to the housing. The drive mechanism, described in more detail below, drives the rotation of the drive train, which drives the rotation of the blades. The drive train disposed in the housing can also be considered part of the drive mechanism when viewed from the perspective of the blades. Drive mechanism coupler 105 translates a rotational force applied to the coupler by the drive mechanism (not shown) to the drive train disposed within housing 101.

FIG. 1 also shows release holes 111-115 which allow for removal of sacrificed material during formation of the working end.

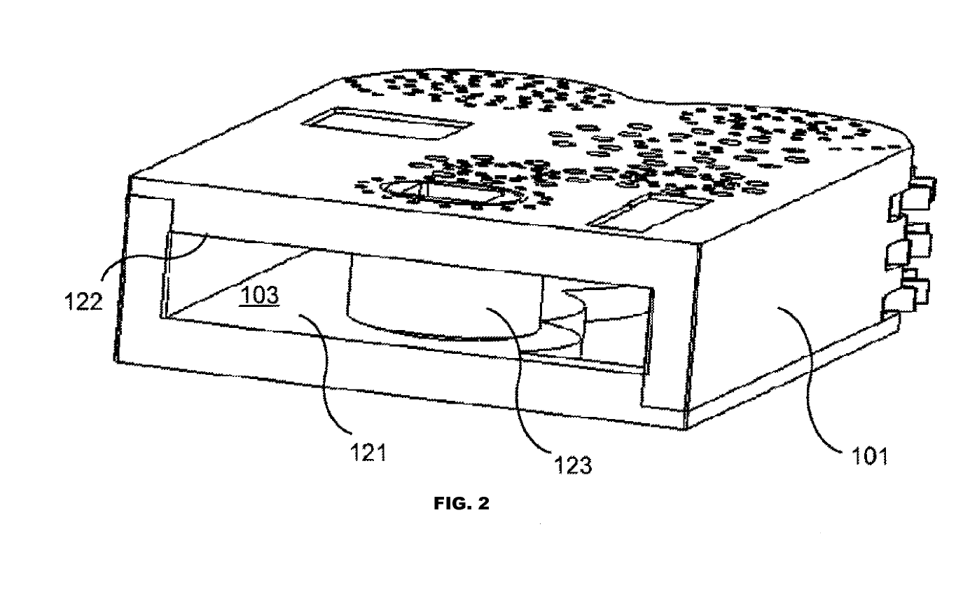

FIG. 2 shows a perspective view of the proximal end of tissue removal device working end 100. Material directed into housing 101 by the rotating blades is directed into chamber 103, wherein it can be stored temporarily or directed further proximally, as described below. A first gear train cover 121 provides for a first surface of chamber 103, while a second gear train cover 122 provides a second surface of chamber 103. FIG. 2 also shows drive mechanism coupler cover 123.

In some embodiments in which the working end 100 includes a storage chamber, the chamber may remain open while in other embodiments it may be closed while in still other embodiments it may include a filter that only allows passage of items of a sufficiently small size to exit.

FIG. 3 shows a perspective view of the distal end of the working end 100. In this embodiment the blades in stack 102 are interdigitated with the blades in stack 104 (i.e. the blade ends are offset vertically along dimension H and have maximum radial extensions that overlap laterally along the width dimension W. The blades can be formed to be interdigitated by, e.g. if formed using a multi-layer, multi-material electrochemical fabrication technique, forming each blade in stack 102 in a different layer than each blade in stack 104. If during formation portions of separately moveable blade components overlap laterally, the overlapping blades should not just be formed on different layers but should be formed such an intermediate layer defines a vertical gap between them. For example, the bottom blade in stack 102 is shown formed in a layer beneath the layer in which the bottom blade in stack 104 is formed.

When manufacturing tissue removal devices of the various embodiments set forth herein using a multi-layer multi-material electrochemical fabrication process, it is generally beneficial if not necessary to maintain horizontal spacing of component features and widths of component dimensions remain above the minimum feature size. It is important that vertical gaps of appropriate size be formed between separately movable components that overlap in X-Y space (assuming the layers during formation are being stacked along the Z axis) so that they do not inadvertently bond together and to ensure that adequate pathways are provided to allow etching of sacrificial material to occur. For example, it is generally important that gaps exist between a gear element (e.g. a tooth) in a first gear tier and a second gear tier so that the overlapping teeth of adjacent gears do not bond together. It is also generally important to form gaps between components that move relative to one another (e.g., gears and gear covers, between blades and housing, etc.). In some embodiments the gaps formed between moving layers is between about 2 um and about 8 um.

In some embodiments, it is desired to define a shearing thickness as the gap between elements has they move past one another. Such gaps may be defined by layer thickness increments or multiples of such increments or by the intralayer spacing of elements as they move past one another. In some embodiments, shearing thickness of blades passing blades or blades moving past interdigitated fingers, or the like may be optimally set in the range of 2-100 microns or some other amount depending on the viscosity or other parameters of the materials being encountered and what the interaction is to be (e.g. tearing, shredding, transporting, or the like). For example for shredding or tearing tissue, the gap may be in the range of 2-10 microns, or in some embodiments in the range of 4-6 microns.

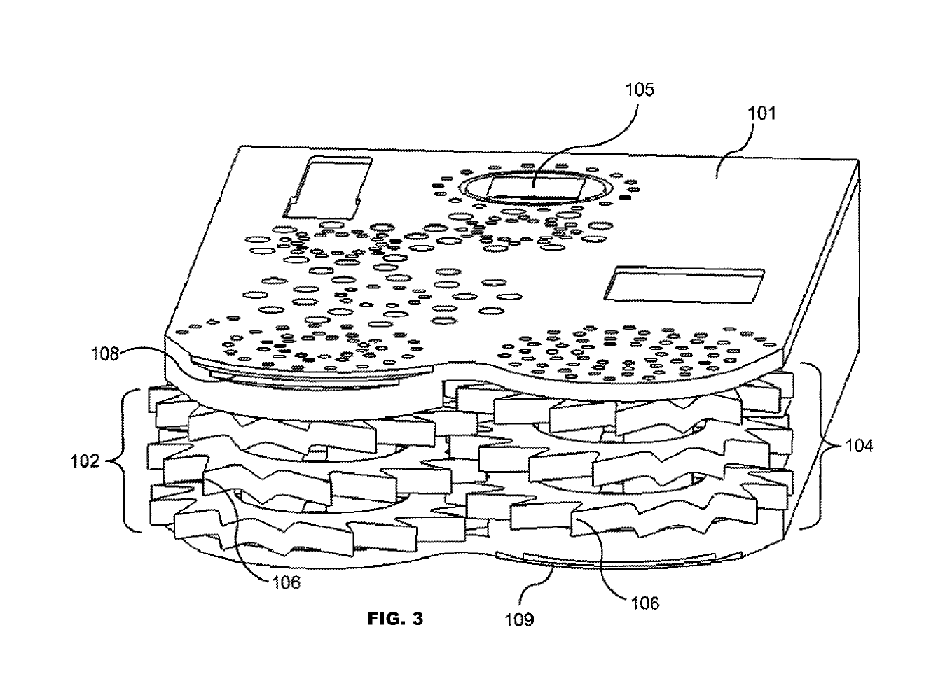

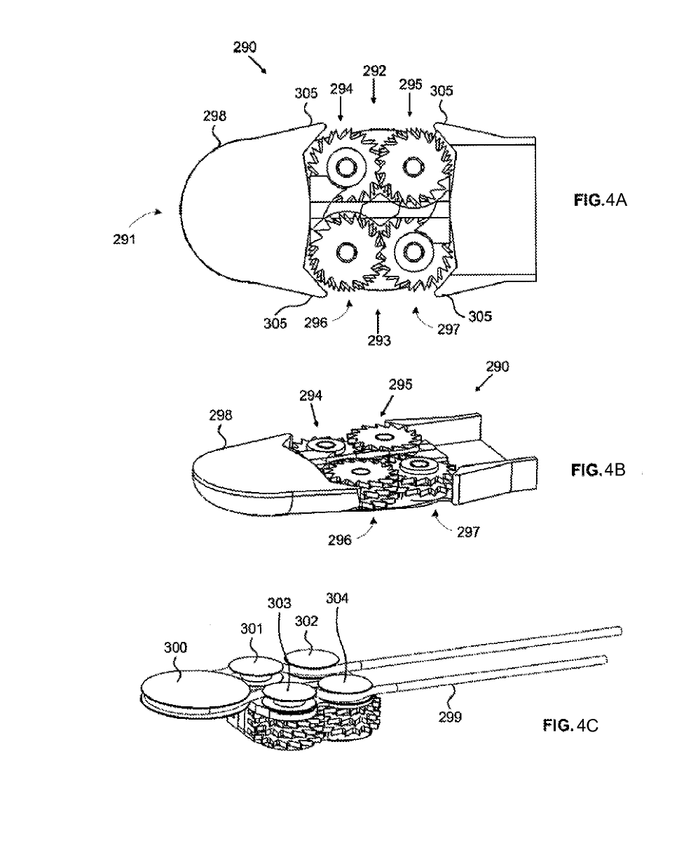

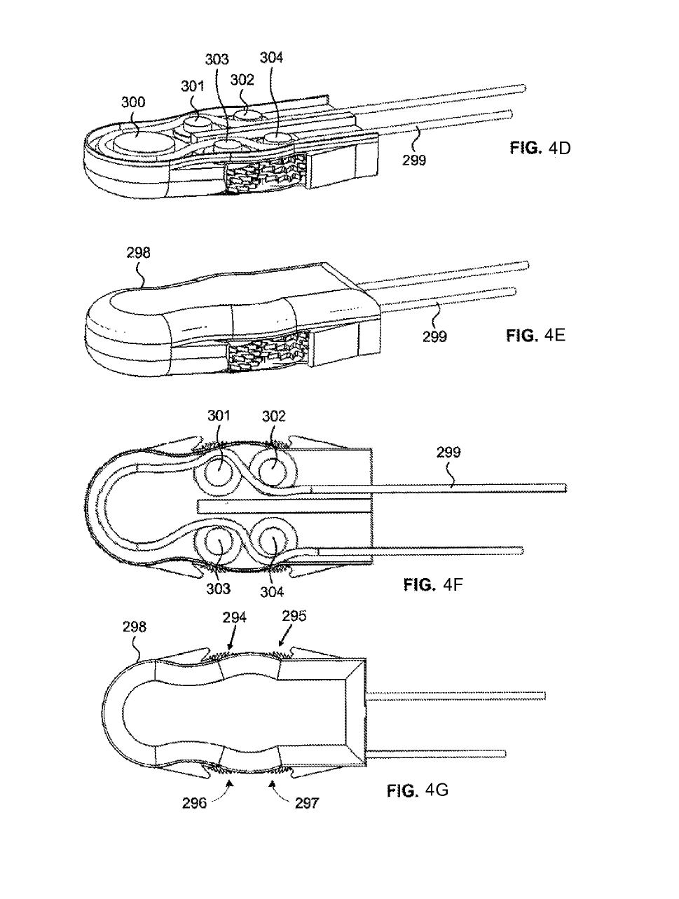

FIGS. 4A-4G illustrate an example a of a side tissue removal working end. FIG. 4A is a top sectional view with a top portion of the housing removed, which shows working end 290 comprising housing 298 and four tissue removal elements 294-297, which are shown as blade stacks. Blade stacks 294 and 295 process tissue along one side of the housing by directing tissue in the direction of arrow 292. Blade stacks 296 and 297 process tissue along a second side of the housing by directing tissue in the direction of arrow 293. As shown in FIGS. 4A-B, blade stacks 294 and 297 each have two blades, while blade stacks 295 and 296 each have three blades. FIG. 4C shows a perspective view without housing 298 illustrating the drive mechanism for the side tissue removal device 290. The drive mechanism includes belt 299, distal pulley 300, and side pulleys 301-304. The side pulleys are coupled to the blade stacks and rotation of the side pulleys rotates the blade stacks. The belt is disposed through side pulleys 301 and 302 and around distal pulley 300 before returning through side pulleys 303 and 304. Actuating of belt 299 therefore activates all four blade stacks. In some embodiments the belt is a nitinol wire, but can be any other suitable material. FIG. 4D is a view with the top portion of the housing removed to show the internal drive mechanism. FIG. 4E shows the same view with the top on the housing. FIGS. 4F and 4G show top views of the working end shown in FIGS. 4D and 4E, respectively. Vacuum, irrigation, or a combination of the two may be used to send extracted tissue from the interior of the working end, proximally to a storage reservoir (e.g. within the working end or located outside the body of the patient on which a procedure is being performed).

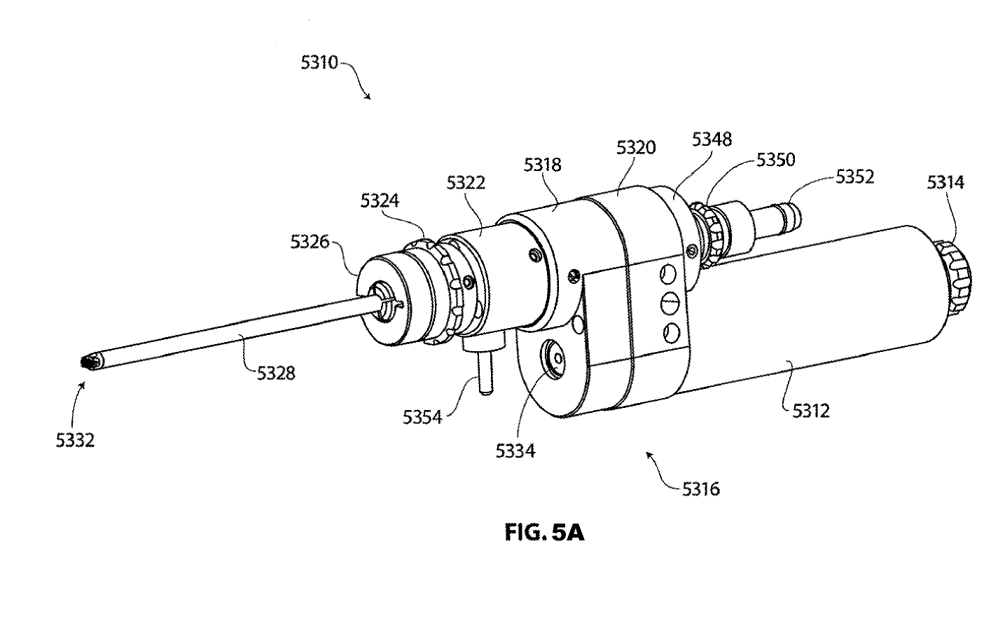

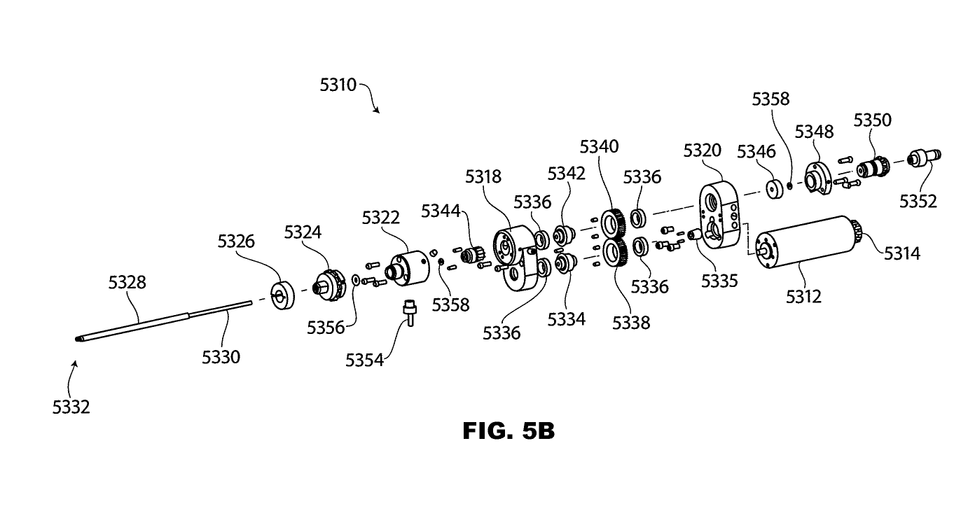

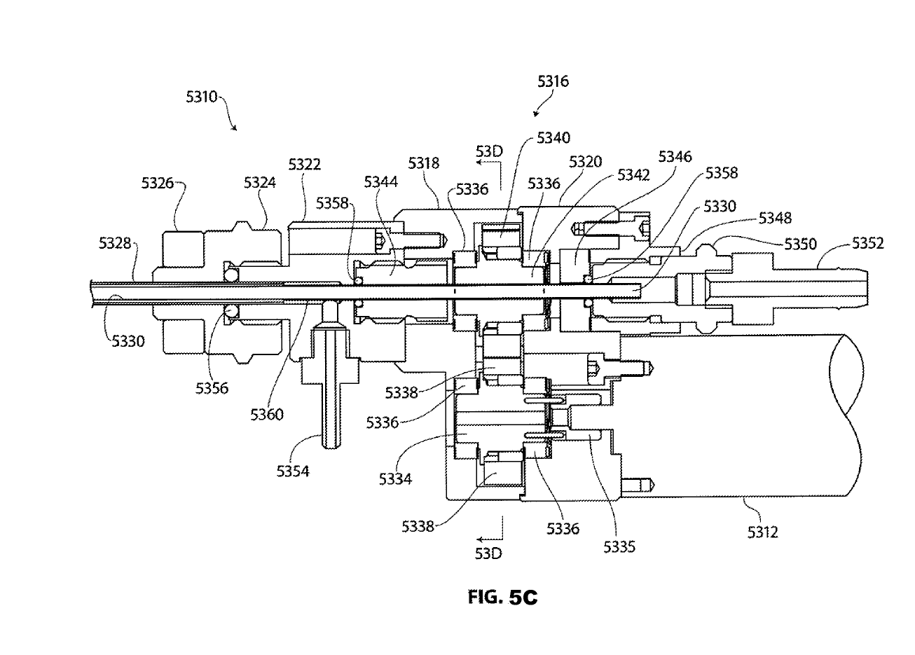

FIGS. 5A-5C show another exemplary embodiment of a tissue removal device. Device 5310 may employ any of the cutting heads described herein, or other suitable cutting heads. In some embodiments, a double rotor shredding head is employed at the distal end of device 5310 to selectively debride tissue down to the cellular level.

In this exemplary embodiment, handheld device 5310 includes a stepper motor 5312 at its proximal end. In other embodiments, other types of electric, pneumatic or hydraulic motors, servos, or other prime movers may be used. The proximal end of motor 5312 may be provided with a manually turnable thumbwheel 5314, as shown. In this embodiment, the distal output end of motor 5312 is provided with a housing 5316, which is made up of a front cover 5318 and a rear cover 5320. Located distally from housing 5316 are an outer shaft housing 5322, an outer shaft lock seal 5324, and a support clamp 5326. A non-rotating, outer support tube 5328 extends from within the proximal end of device 5310 towards the distal end of the device. Within support tube 5328, a rotating drive tube 5330 (best seen in FIGS. 5B and 5C) also extends from within the proximal end of device 5310 towards the distal end of the device. The support tube 5328 and inner drive tube 5330 may collectively be referred to as an introducer. A cutter head assembly 5332, subsequently described in detail, is attached to the distal end of support tube 5328.

As best seen in FIG. 5B, other components of device 5310 include motor shaft drive axle 5334, motor dog 5335, four bearings 5336, drive gear 5338, driven gear 5340, inner drive shaft axle 5342, inner shaft lock seal 5344, vacuum gland disk 5346, vacuum seal lock housing 5348, vacuum seal lock 5350, vacuum hose barb 5352, irrigation fluid hose barb 5354, outer tube o-ring 5356, and two vacuum gland o-rings 5358. Various other pins, dowels, fasteners, set screws, ball detents, shims and wave disc springs are shown in the figures without reference numerals. As will be appreciated by those skilled in this art, these non-referenced components serve to align, retain and ensure the proper functioning of the other components of exemplary device 5310.

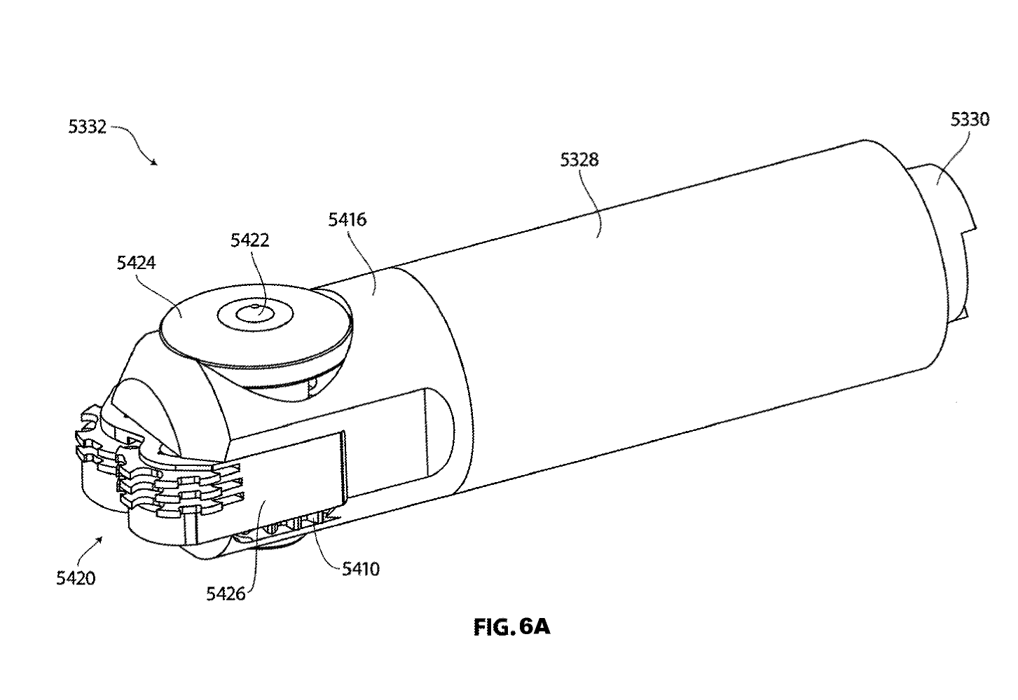

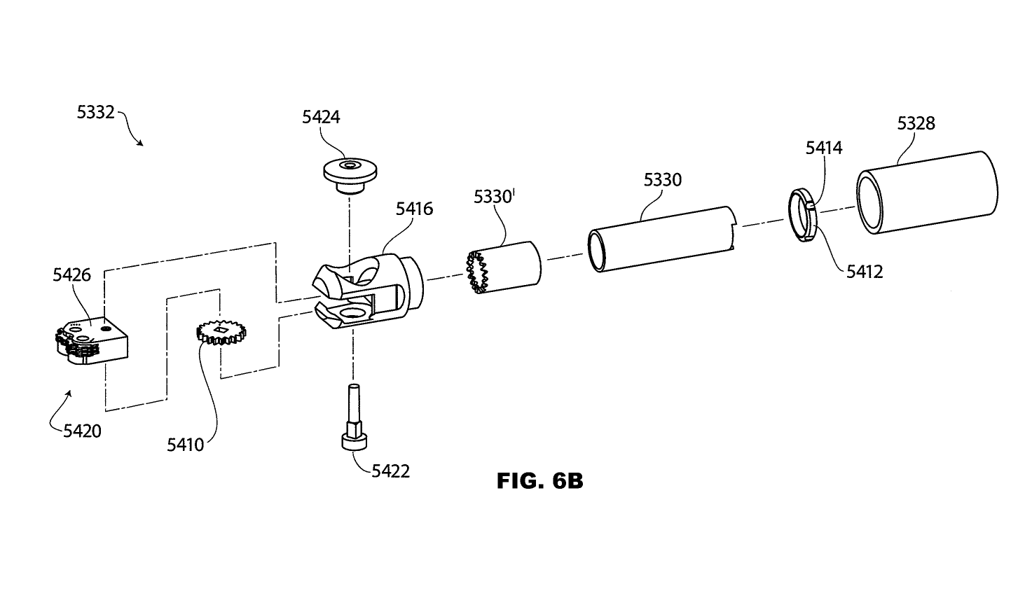

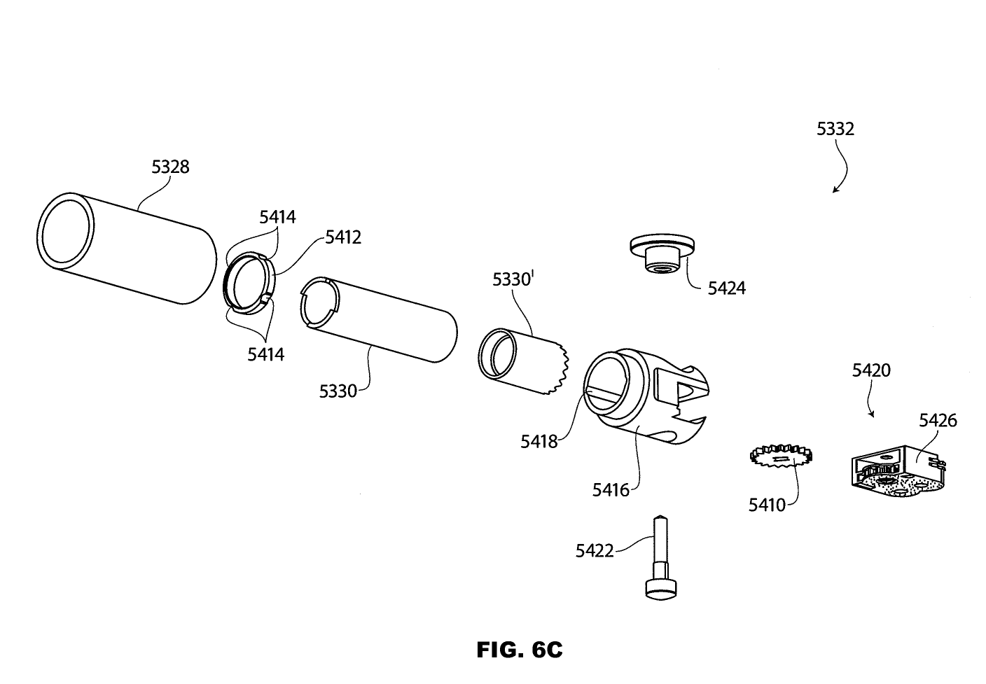

The two rotors of cutter head assembly 5332 located at the distal end of device 5310 are driven by motor 5312 through drive tube 5330 and other drive components of device 5310, as will now be described in more detail. As best seen in FIGS. 5B and 5C, a motor dog 5335 is attached to the output shaft of motor 5312. Motor dog 5335 is coupled to motor shaft drive axle 5334, which is rotatably mounted in housing 5316 with two bearings 5336. Drive gear 5338 is rigidly fixed to motor shaft drive axle 5334, and drives driven gear 5340. Driven gear 5340 is rigidly fixed to inner drive shaft axle 5342, which is rotatably mounted in housing 5316 with two bearings 5336. Inner rotating drive tube 5330 passes through the center of inner drive shaft axle 5342 and is rotatably fixed thereto. Drive tube 5330 extends from the proximal end of device 5310 to the distal end of the device through the non-rotating outer support tube 5328. The distal end of drive tube 5330 (or a separate tube 5330′ attached thereto) is provided with crown teeth around its periphery, as shown in FIGS. 6B and 6C, for meshing with drive gear 5410. As drive tube 5330 is rotated about a longitudinal axis of device 5310 by motor 5312 through the above-described drive train components, it drives drive gear 5410 about an axis that is perpendicular to the longitudinal axis, as can be appreciated by viewing FIG. 6. Drive gear 5410 in turn drives other components of the cutter head assembly, and as is subsequently described in more detail.

In some embodiments motor 5312 is provided with feedback control for rotational velocity and torque. These two parameters can be used for controlling and monitoring changes in rotational velocity and the torque load. For measuring rotational velocity, an encoder may be located at one or more of the cutter rotors, at the drive motor, or at another location along the drive train between the drive motor and cutter rotors. In some embodiments, the encoder is located at or close to the rotors to avoid backlash associated with the drive train, thereby making the velocity monitoring more responsive and accurate. Encoder technologies that may be used include optical, resistive, capacitive and/or inductive measurement. To sense torque load, one or more strain gages may be located at the cutter rotors, at the drive motor, or at another location along the drive train between the drive motor and cutter rotors. Torque load may also be sensed by monitoring the current being drawn by the motor. By sensing changes in velocity and/or torque, a controller associated with device 5310 can determine that the cutter rotors are passing from one tissue type to another and take appropriate action. For example, the controller can sense when the cutter elements are passing from soft to hard tissue, from hard to medium density tissue, or from a cutting state to non-cutting state. In response to these changes, the controller and/or device 5310 can provide audio, visual and/or tactile feedback to the surgeon. In some embodiments, the controller can change the velocity, direction or stop cutter rotors from rotating in response to velocity and/or torque feedback. In one embodiment of the invention, a typical cutting rotor speed is on the order of 100 to 20,000 rotations per minute, and a typical torque load is on the order of 0.25 to 150 mN-meter. Other sensors, such as a pressure sensor or strain sensor located at the distal tip of device 5310, may also be utilized to provide feedback that tissue cutting elements are moving from one tissue type to another. In some embodiments, an impendence sensor may be located at the distal tip of the device, to sense different tissue types or conditions, and provide corresponding feedback for tissue cutting control when the tissue being cut by the cutter head changes. Such a pressure sensor feedback control arrangement can be used with types of cutting devices other than those disclosed herein.

Referring now to FIG. 5C, irrigation fluid hose barb 5354 is provided on the lower side of outer shaft housing 5322 of exemplary device 5310. Hose barb 5354, or a similar fluid line coupling, may be connected to a supply of irrigation fluid. The lumen of hose barb 5354 is in fluid communication with an internal irrigation fluid cavity 5360. Fluid cavity 5360 surrounds internal drive tube 5330, and is bounded on its proximal end by o-ring seal 5358 around drive tube 5330. Fluid cavity 5360 is bounded on its distal end by o-ring seal 5356 around outer support tube 5328. This arrangement allows drive tube 5330 to rotate, but constrains irrigation fluid delivered from hose barb 5354 to travel only through the annular space defined by the outer surface of drive tube 5330 and the inner surface of support tube 5328. Irrigation fluid may thus flow distally through the annular space to the distal end of device 5310.

As shown in FIG. 6B, one or more drive aligner rings 5412 may be provided between outer support tube 5328 and inner drive tube 5330 along their lengths to support drive tube 5330 as it rotates. In order to allow the flow of irrigation fluid between the tubes 5328 and 5330, rings 5412 may be provided with one or more channels 5414 as shown. When the distal flow of irrigation fluid reaches the cutter head assembly 5332, it continues to flow distally into lug 5416. To enable the fluid flow, lug 5416 is provided with fluid channels 5418 located along the outer walls of its central bore, as best seen in FIG. 6C. In this embodiments, irrigation fluid passes distally between inner drive tube 5330 and lug 5416 through channels 5418 (only one channel shown in FIG. 6C). Irrigation fluid flowing distally through channels 5418 may be directed toward the outside portions of cutting elements. In this embodiment, the outside portions of cutting elements are rotating distally, away from the fluid flow, while the inside portions of cutting elements are rotating proximally, toward the center of lug 5416 and drive tube 5330.

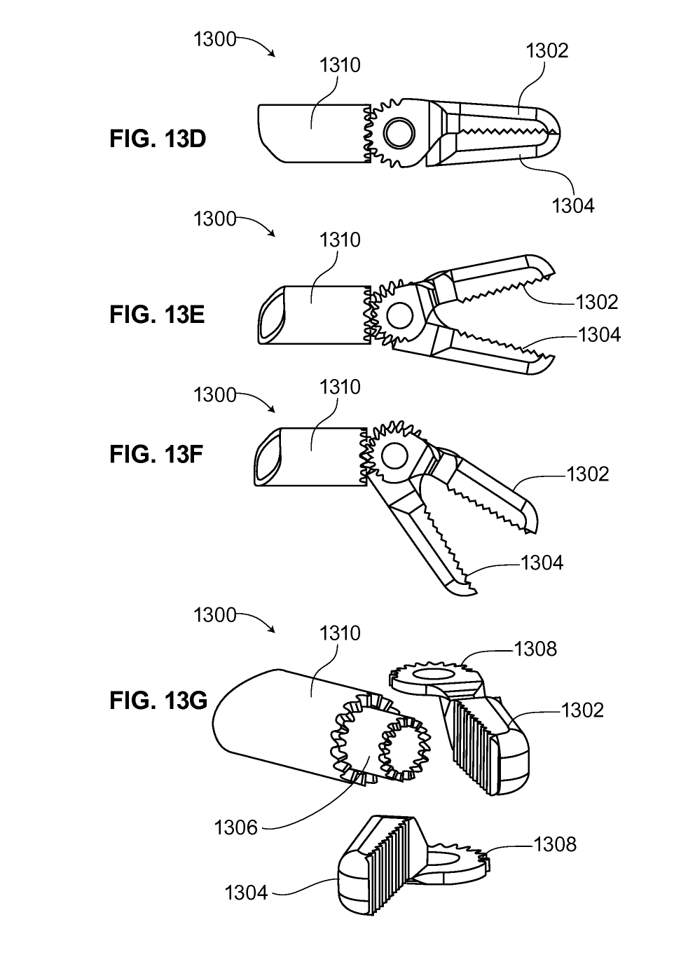

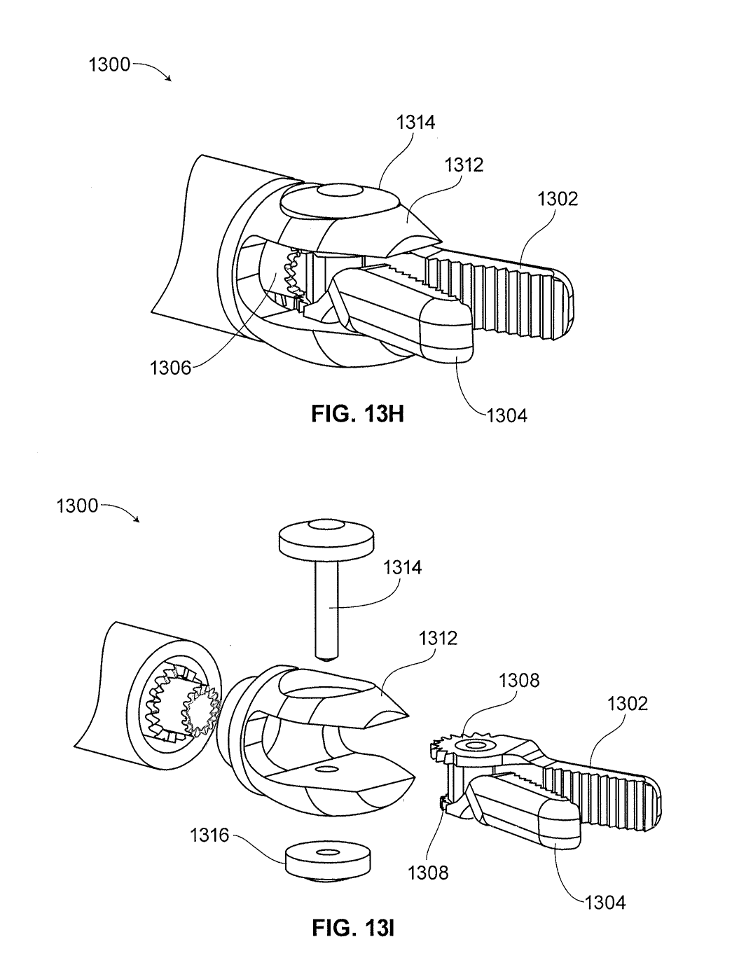

In some embodiments, the irrigation fluid serves multiple functions. The irrigation fluid can serve to lubricate the cutting elements, drive gears, journal bearings and other components as the parts rotate. The irrigation fluid can also serve to cool the cutting elements and/or the tissue being cut, absorbing heat and carrying it away as the irrigation fluid is removed from the patient. The fluid can serve to flush tissue particles from the moving parts to prevent them from becoming clogged. The fluid can also serve to carry away the tissue portions being cut and remove them from the target tissue site. In some embodiments, the irrigation fluid is ple, tissue grasping device 1300 shown in FIGS. 13A-13I may have an electrode located on the distal housing or lug 1312, or tWith the current exemplary cutting device 5310, however, the irrigation fluid and/or other bodily fluids may be removed from the target tissue site by the cutting device 5310, as will now be described in detail.

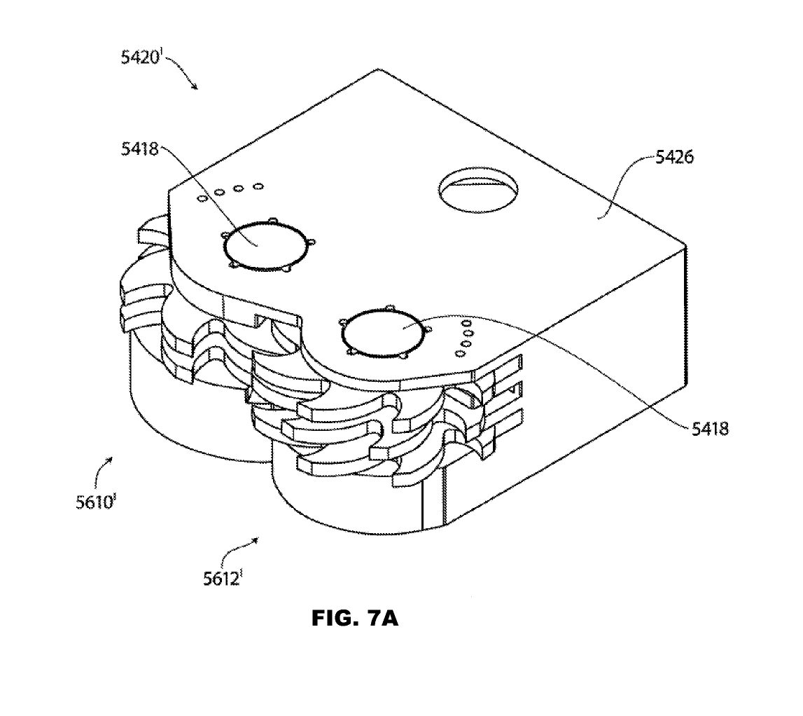

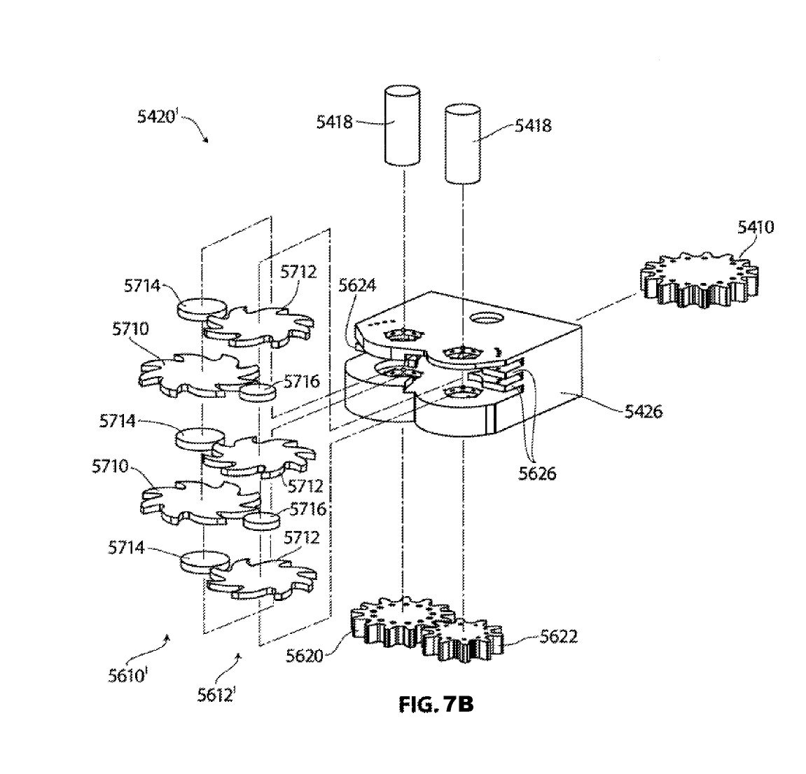

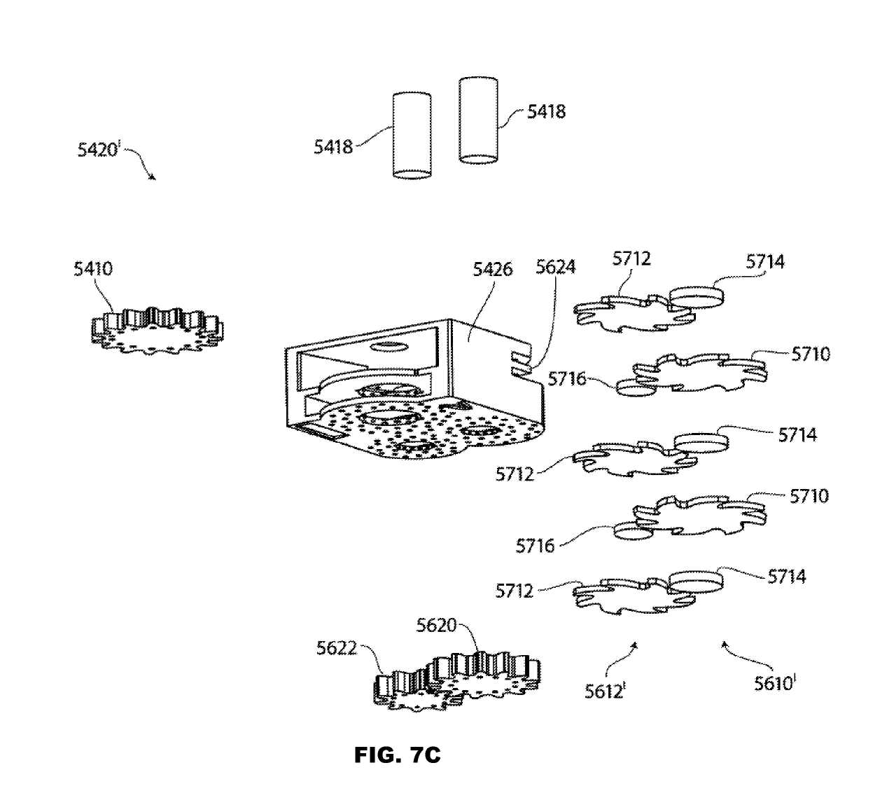

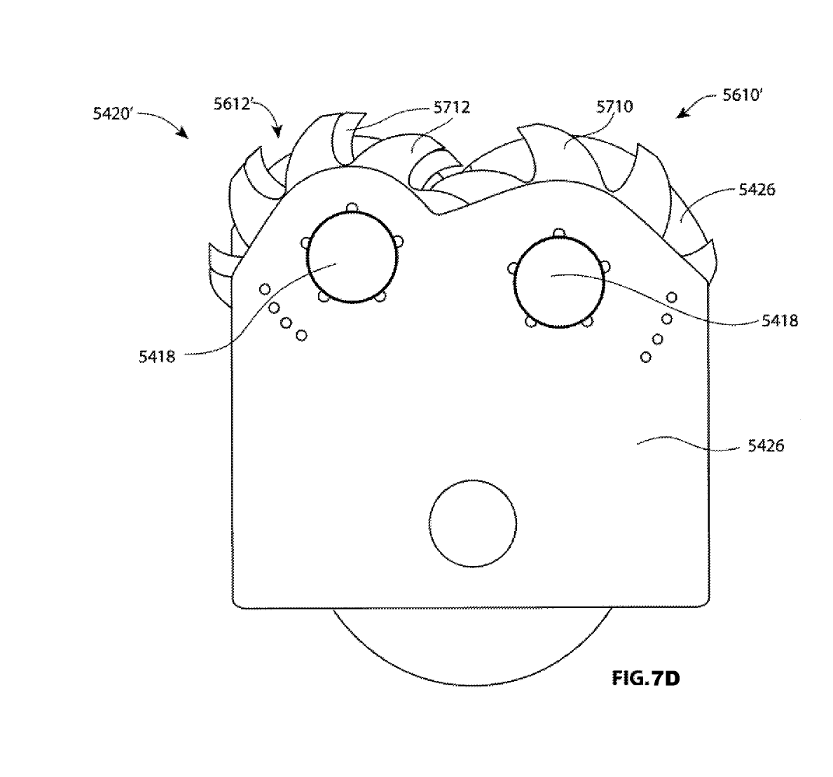

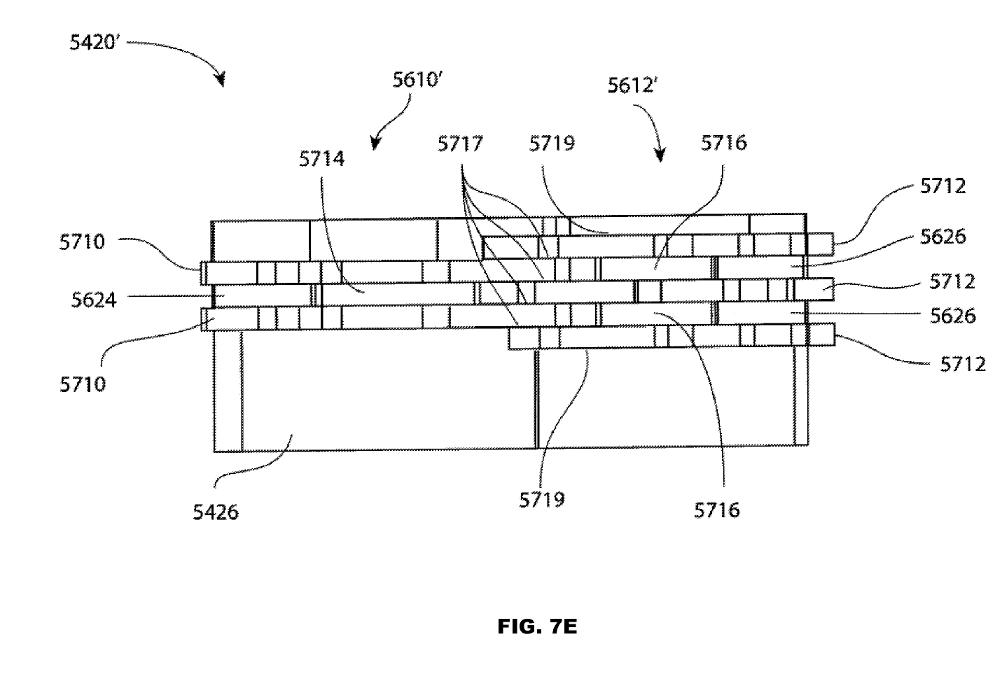

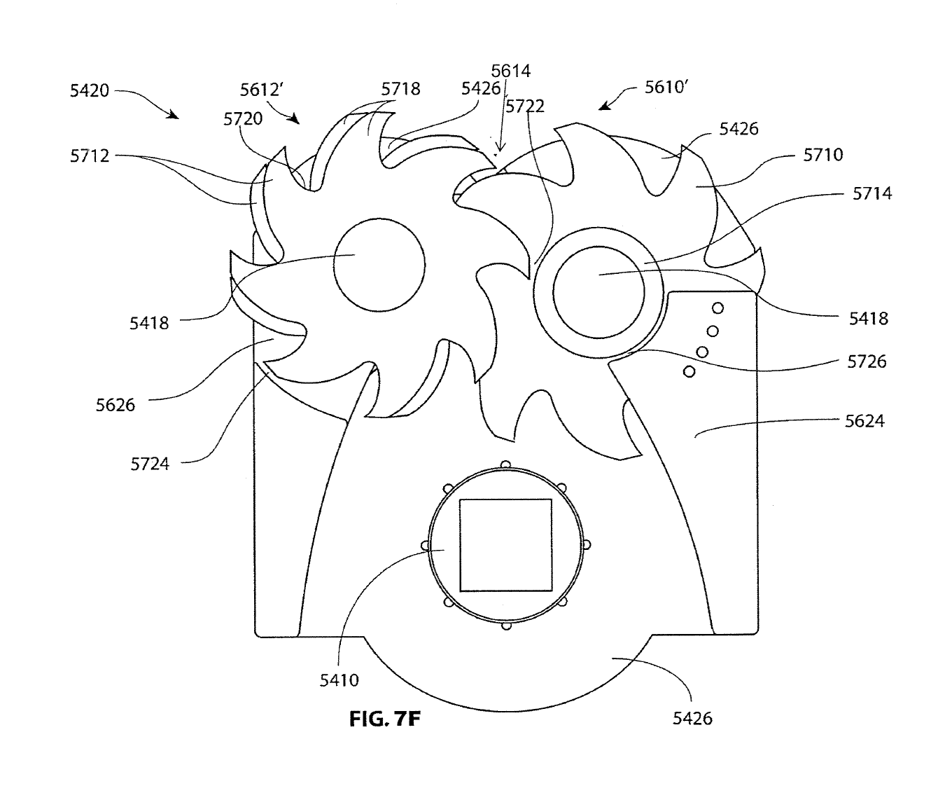

As previously described, irrigation fluid may be delivered to cutting elements and/or a target tissue site through device 5310. Exemplary device 5310 is also constructed to remove the irrigation fluid and tissue portions cut from the target tissue site through the shaft of device 5310. As can be appreciated by viewing FIG. 7F, the two interleaving stacks of cutting elements, also referred to as rotors 5610 and 5612, have an overlapping section 5614 in the center of cutter head assembly 5332. The two rotors 5610 and 5612 may be rotated in opposite directions such that each rotor engages target tissue and pulls it towards the central overlapping section 5614. In overlapping section 5614, the tissue is shredded into small pieces by the interdigitated cutting elements, as is subsequently described in more detail. The small tissue portions are generally propelled in a proximal direction by rotors 5610 and 5612, away from the target tissue site and into the cutter head assembly 5332. As can be appreciated by viewing FIG. 7F, the shredded tissue portions emerge from rotors 5610 and 5612 substantially along the central axis of lug 5416 (and therefore also the central axis of drive tube 5330. With sufficient irrigation fluid being supplied to the tissue cutting area, and sufficient aspiration being provided from the proximal end of the device, irrigation fluid around rotors 5610 and 5612 carries the cut tissue particles proximally down the center of drive tube 5330. As shown in FIG. 5C, the proximal end of drive tube 5330 is in fluid communication with hose barb 5352 located at the proximal end of device 5310. A traditional aspiration device or other suction source may be attached to device 5310 through hose barb 5352 or other suitable fluid coupling to collect the spent irrigation fluid and cut tissue portions.

In some embodiments, the cut tissues portions emerging from hose barb 5352 may be collected for testing. The tissue portions may be separated from the irrigation fluid, such as by centrifugal force, settling and/or filtering. The tissue portions may be measured to precisely determine the mass and/or volume of tissue removed. The pathology of some or all of the tissue portions may also be determined. In some embodiments, the above testing may be performed during a surgical procedure so that results of the testing may be used to affect additional stages of the procedure.

According to aspects of the invention, the inside diameter of drive tube 5330 may be much larger than the maximum dimension of the tissue portions traveling through it. In some embodiments, the maximum tissue dimension is less than about 2 mm across. In one exemplary embodiment, the inside diameter of drive tube 5330 is about 3 mm, the outside diameter of the support tube 5328 is about 5.6 mm, and the maximum dimension of the tissue portions is about 150 microns. In another exemplary embodiment, the inside diameter of drive tube 5330 is about 1.5 mm, the outside diameter of the support tube 5328 is about 2.8 mm, and the maximum dimension of the tissue portions is about 75 microns. In other embodiments, the inside diameter of drive tube 5330 is between about 3 mm and about 6 mm. In some embodiments, the maximum dimension of the tissue portions is at least one order of magnitude less than a diameter of the tissue removal lumen. In other embodiments, the maximum dimension of the tissue portions is at least twenty times less than a diameter of the tissue removal lumen. In some embodiments, the maximum dimension of the tissue portions is less than about 100 microns. In other embodiments, the maximum dimension of the tissue portions is about 2 microns.

Referring now to FIGS. 6A-6C, an exemplary cutter head assembly 5332 is described in more detail. Cutter head assembly 5332 may be used with debriding device 5310, shown in FIGS. 6A-6C. As best seen in FIG. 6B, cutter head assembly 5332 includes lug 5416, drive gear 5410, rotor housing assembly 5420, aligner pin 5422, and aligner cap 5424. Lug 5416 is provided with a cutout on its distal end for receiving rotor housing assembly 5420. Beneath the rotor housing cutout, lug 5416 has a circular recess for receiving drive gear 5410. A bore is provided in the bottom of lug 5416 for receiving the head of aligner pin 5422. When cutter head 5332 is assembled, the shank of aligner pin 5422 passes through the bore of lug 5416, through a square aperture in the center of drive gear 5410, through a bore in the proximal end of rotor housing assembly 5420, and into a large diameter bore through the top of lug 5416. Aligner cap 5424 is received with the large diameter bore in the top of lug 5416, and is fastened to aligner pin 5422 by a press fit, weld, threads, a separate fastener, or other suitable means. In this assembled arrangement, pin 5422 and cap 5424 retain rotor housing 5426 from moving longitudinally relative to the central axis of the instrument, and rotor housing 5426 and drive gear 5410 retain pin 5422 and cap 5424 from moving radially relative to the central axis of the instrument. Pin 5422 and cap 5424 spin together as a unit relative to lug 5416, and serve to align drive gear with the distal end of drive tube 5330′, as previously described. Pin 5422 also serves to transmit torque from drive gear 5410 to gear 5616, which resides inside the rotor housing directly above drive gear 5410. Lug bearing 5416 forms the base of cutter head assembly 5332, shown in FIGS. 6A-6C. As subsequently described in further detail, various different cutter heads may alternately be inserted into and secured within the slot shaped opening in the distal end of the lug bearing.

FIGS. 7A-7F show further details of an exemplary rotor housing assembly 5420′. Assembly 5420′ is constructed and operates in a manner similar to assembly 5420 as previously described in reference to FIGS. 6A-6C, but has a different blade configuration. As shown in FIG. 7A, rotor housing assembly 5420′ includes a pair of rotors 5610′ and 5612′, each rotatably mounted in rotor housing 5426 by an axle 5618. In this embodiment, rotors 5610′ and 5612′ are configured to rotate in opposite directions to draw tissue into a center, overlapping region where the tissue is shredded.

Referring to FIGS. 7B and 7C, the components of rotor housing assembly 5420′ are shown. Assembly 5420′ includes housing 5426, a pair of axles 5418, and gears 5410, 5620 and 5622, as previously described. Rotor 5610′ includes two blades 5710 interspersed with three spacer rings 5714 on first axle 5418. Rotor 5612′ includes three blades 5712 interspersed with two spacer rings 5716 on second axle 5418.

It should be noted that while rotor housing assembly 5420′ is shown in an exploded format for clarity in FIGS. 7B and 7C, suggesting that the components are fabricated separately and then assembled using traditional assembly processes, this may or may not be the case, depending on the embodiment. In some embodiments, rotor assembly 5420′ is assembled this way. In other embodiments, assembly 5420′ may be built in layers, such as by using a MEMS fabrication processes. For example, after portions of housing 5426 and gears 5410, 5620 and 5622 are built up in layers, bottom blade 5712, bottom spacer 5714, and housing fin 5624 are formed together in one or more layers. Following this layer, bottom blade 5710, bottom spacer 5716, and bottom housing fin 5626 may be formed together in one or more layers. The process may be repeated until the entire rotors 5610′ and 5612′ and surrounding components are formed. A thin sacrificial layer may be formed between adjacent layers of components to separate the components from one layer from components of adjacent layers. Sacrificial material may also be formed in portions of each non-sacrificial layer to separate components on that layer, create desired voids in the finished assembly, and to provide a substrate for forming components in subsequent layers above. With such a fabrication technique, rotor 5610′ may be formed as a single unitary structure interleaved with portions of rotor housing 5426, rather than separate components (i.e. axle 5418, spacers 5714, blades 5710, and gear 5620.) Similarly, rotor 5612′ may be formed as a single unitary structure interleaved with portions of rotor housing 5426, rather than separate components (i.e. axle 5418, blades 5712, spacers 5716, and gear 5622.) In some embodiments, combinations of fabrication and assembly techniques may be used to create the rotor housing and/or cutter head assemblies.

Referring to the top view shown in FIG. 7D, it can be seen that in this embodiment the axle 5418 of rotor 5612′ is more distally located than axle 5418 of rotor 5610′. It can also be seen that while a top plate portion of rotor housing 5426 covers most of rotor blades 5710 and 5712, the blades protrude less from a middle and bottom plate portion of housing 5426. Further details of protruding blades and rotor characteristics are subsequently discussed in reference to FIG. 7F.

A front or distal end view is shown in FIG. 7G. As depicted in FIG. 7G, very small gaps or interference fits 5717 between overlapping blades 5710 and 5712 are desirable in some embodiments. Similarly, very small gaps or interference fits 5719 between blades 5712 and adjacent portions of rotor housing 5426 are desirable in some embodiments, as will be subsequently described in more detail.

Referring to the cross-sectional plan view of FIG. 7F, the bottom two blades 5712 of rotor 5612′ and the bottom blade 5710 of rotor 5610′ are shown. As shown, blades 5710 have a larger outer diameter than that of blades 5712. But because axle 5418 of rotor 5612′ is located more distally than axle 5418 of rotor 5610′, blades 5712 protrude more distally from the bottom of rotor housing 5426 than do blades 5710 of rotor 5610′. It can also be seen that teeth 5718 and associated troughs 5720 of blades 5712 are configured to be rotationally out of phase with those of other blades 5712 of rotor 5612′. As will subsequently be discussed in more detail, this arrangement can tune rotors 5612 to selective cut certain types of tissue and avoid cutting other types of tissue.

Various rotor gaps can be seen in FIG. 7F. For example, gap 5722 is shown between the tips of blade teeth 5718 of rotor 5612′ and spacer ring 5714/axle 5418 of opposing rotor 5610′. Gap 5724 is also shown, between the tips of blade teeth 5718 of rotor 5612′ and the adjacent portion of housing 5426. Gap 5726 is also shown, between spacer ring 5714/axle 5418 of rotor 5610′ and the adjacent portion of housing 5426. In some embodiments, it is desirable to keep gaps 5722, 5724 and 5726 very small, to ensure that tissue portions/particles that pass through rotors 5610′ and 5612′ are first cut to a very small size, and to avoid jamming or clogging rotors 5610′ and 5612′. In some embodiments, these gaps are fabricated as small interferences between the adjacent parts so that when the rotors are first rotated, the adjacent parts hit each other and wear down or burnish each other. In this manner, after a break in period, smaller interference or zero clearance fits are created between the adjacent moving parts. Gap distances that applicants believe are advantageous include less than about 20 microns, less than about 10 microns, less than about 5 microns, less than about 1 micron, substantially zero, an initial interference fit of at least 2 microns, and an initial interference fit of about 5 microns.

In operation, the cutter elements of rotor housing assembly shown in FIGS. 7A-7F serve to grab tissue from a target source, draw the tissue towards a central region between the blades, cut the tissue from the source, and morcellate the tissue in small pieces for transport away from the body. In other embodiments, separate cutter elements may be used for these various functions. For example, one blade or blades may be used to cut tissue from the source, while another blade or set of blades may be used to morcellate the cut tissue.

Components of cutter head assembly 5332, including rotor housing assemblies 5420 and 5420′, may be fabricated using processes such as laser cutting/machining, photo chemical machining (PCM), Swiss screw, electro-discharge machining (EDM), electroforming and/or other processes for fabricating small parts. Wafer manufacturing processes may be used to produce high precision micro parts, such as EFAB, X-ray LIGA (Lithography, Electroplating, and Molding), and/or UV LIGA. An electrochemical fabrication technique for forming three-dimensional structures from a plurality of adhered layers is being commercially pursued by applicant Microfabrica® Inc. (formerly MEMGen Corporation) of Van Nuys, Calif. under the name EFAB®. Such a technique may be advantageously used to fabricate components described herein, particularly rotors and associated components.

In some embodiments, the shredder's ability to selectively remove tissue is attributed to the protrusion of the rotating cutters from the housing and the design of a tooth pitch (space between the tips of adjacent teeth) of each rotor. In some embodiments, the protrusion sets the depth of the inward cut for the tips of the rotor. This inward depth controls the thickness of tissue being removed. The tooth pitch or number of teeth circumferentially about the rotor diameter provides an opening for individual tissue fibers and/or fiber bundles to be hooked, tensioned and drawn between the cutters.

From the point of view of the selected tissue, the tooth pitch and protrusion may be designed to grasp the smallest fibers or fiber bundles that are to be removed. From the point of view of the non-selected tissue, the tooth pitch may be many times smaller than the fiber or fiber bundle, and the protrusion may also be equally smaller than the fiber/bundle diameter.

As previously described, FIG. 7D shows the exemplary protrusion of blades 5710 and 5712 as viewed from the top of a rotor housing assembly 5420′. In some embodiments, the protrusion is more exposed on the top side than the bottom. In other embodiments, the cutter device has the same protrusion for both sides. Biasing the protrusion more on one side than the other can provide advantages such as cutting/shredding directionality and/or additional safety. Blade protrusion distances that applicants believe are advantageous include less than about 100 microns, less than about 10 microns, substantially flush with the housing, recessed a minimum of about 5 microns, and recessed a minimum of about 10 microns.

Tooth pitch is the distance from one tooth tip to the next tooth tip along an imaginary circle circumscribing the outer circumference of the blade. The trough diameter or depth generally is the distance between the tooth tip and the low point between the tooth tips. In many embodiments, the trough is a critical geometry component that enables tissue selectivity. Additionally, the trough opening (i.e. the distance from tooth tip to the tooth back of an adjoining tooth) can determine the size of the “window” for capturing a fiber or fiber bundle diameter.

In some embodiments, the target tissue being cut is hydrated and generally has a nominal fiber diameter of about 6 to about 9 microns. In some embodiments, the target tissue being cut is dry and generally has a nominal fiber diameter of about 5 to about 6 microns. In some embodiments, the tissue fibers are connected together in bundles having a nominal diameter of about 250 microns.

Typical dimensions in some embodiments include:
Housing diameter: 6 mm or less
Blade diameter range: 0.75 mm to 4 mm
Tip to Tip range: 0.2 mm to 1 mm
Trough diameter range: 2 microns to 0.5 mm
Blade protrusion range: 2 microns to 2 mm
   The tip to tip distance is typically at least two times the trough diameter for hook type teeth.

The tissue cutting devices disclosed herein may be configured for use in a variety of procedures. An example of a cardiac application is using the inventive devices to selectively remove endocardium, with the cutting device configured to leave the underlying myocardium uncut. An example of a tissue removing application involving the esophagus includes selectively removing mucosa, leaving the submucosa. Such a therapy would be useful for treating Barrett's disease. Examples in the spinal area include selectively removing flavum, with the cutting device configured to stop removing tissue when dura is reached, leaving the dura intact. Selective removal of flavum but not nerve root is another embodiment. A cutting device constructed according to aspects of the invention can also be configured to remove flavum without cutting bone. In this embodiment, the rotor velocity could be changed and/or the cutting elements could be changed after the flavum is removed such that some bone tissue could then be removed. Examples in the neurovascular area include selectively removing cancerous tissue while not cutting adjacent blood vessel tissue or nerve tissue. In the rheumatology field, tears in labral target tissue may be selectively removed while preserving adjacent non-target tissue, such as in the hips, shoulders, knees, ankles, and small joints. In some embodiments, small teeth on the rotors can interact with micron scale fibers of cartilage, removing tissue in a precise way, much like precision machining of materials that are harder than tissue. Other target tissues that may be selectively removed by the inventive devices and methods described herein include cartilage, which tends to be of a medium density, periosteum, stones, calcium deposits, calcified tissue, cancellous bone, cortical bone, plaque, thrombi, blood clots, and emboli.

It can be appreciated by those skilled in the art of tissue removal that soft tissue is much more difficult to remove in a small quantities and/or in a precise way than harder tissue such as bone that may be grinded or sculpted, since soft tissue tends to move or compress when being cut, rather than cut cleanly. Cutting tissue rather than removing it with a laser or other high energy device has the advantage of not overheating the tissue. This allows the tissue to be collected and its pathology tested, as previously described.

In some embodiments of the invention, the selective tissue cutting tool may be moved laterally along a tissue plane, removing thin swaths of tissue with each pass until the desired amount or type of tissue is removed. In some embodiments, the tool may be plunged into the target tissue in a distal direction, until a desired depth or type of tissue is reached. In any of these embodiments, the tool may cut a swath or bore that is as large as or larger than the width of the tool head. In some embodiments, the cutting elements are distally facing, laterally facing, or both.

According to further aspects of the present disclosure, the rotational axis or axes of a single or dual rotor cutter can be located and angled in three-dimensional space in a variety of configurations relative to a longitudinal axis of the debrider device to allow access to target tissue sites not accessible by conventional debriders. These unique configurations enable medical procedures that otherwise could not be performed, or permit the procedures to be performed more easily.

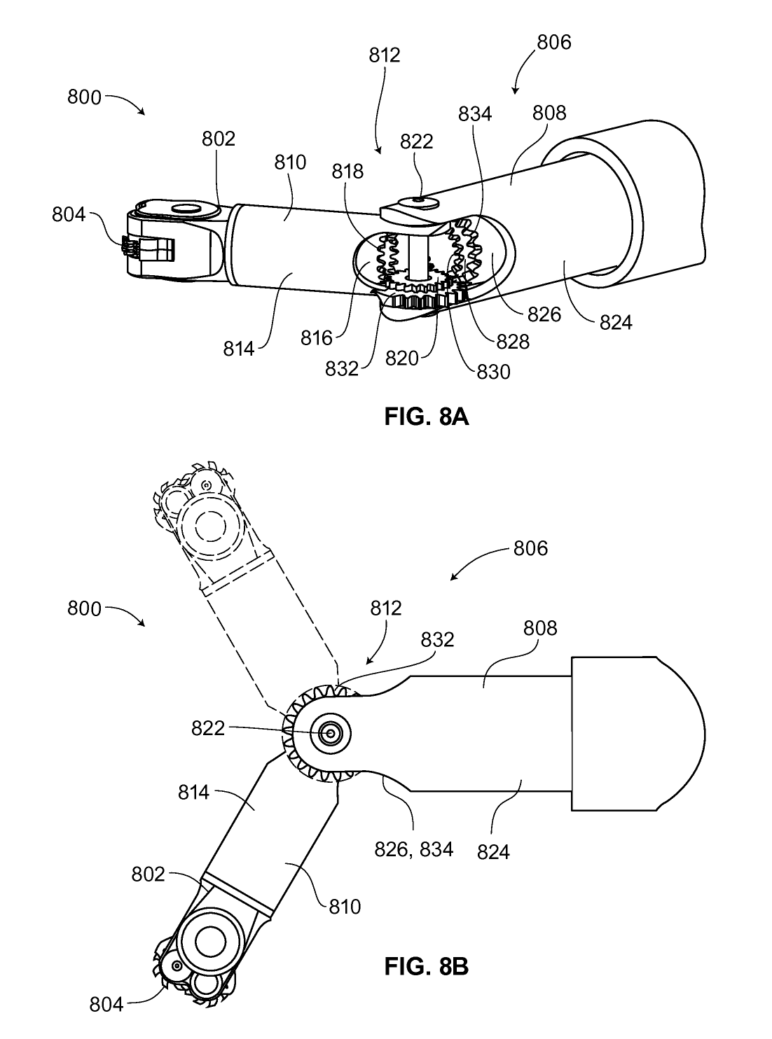

Referring to FIGS. 8A-17, additional embodiments of tissue cutting and manipulating tools are shown that are configured to have one or more degrees of articulation.

Referring first to FIGS. 8A and 8B, an articulating tissue debrider tool 800 is shown. The distal tip of tool 800 has a distal housing or lug 802 configured with a tissue cutter assembly. An elongate member 806 is coupled to the distal housing 802 and is configured to introduce the distal housing 802 to a target tissue site of a subject, as with previously described embodiments. The elongate member 806 comprises a proximal portion 808 having a first central axis therethrough, and a distal portion 810 having a second central axis therethrough. A joint mechanism 812 is provided between the distal end of the proximal portion 808 and a proximal end of the distal portion 810. The joint mechanism 812 is configured to allow the distal portion 810 to articulate with respect to the proximal portion 808, such that the first central axis is non-collinear with the second central axis.

The distal portion 810 of the elongate member 806 includes a distal outer tube 814 and a distal inner drive tube 816 rotatably mounted within the distal outer tube. The distal inner drive tube 816 includes a crown gear at its distal end (not shown) to drive the tissue cutter assembly 804 in a manner similar to previously described embodiments. The distal inner drive tube 816 also includes a crown gear 818 at its proximal end. The crown gear 818 is configured to mesh with a first spur gear 820 of the joint mechanism 812. The first spur gear 820 is rotatably mounted on a spindle 822.

The proximal portion 808 of the elongate member 806 includes a proximal outer tube 824, a proximal inner articulation tube 826 rotatably mounted within the proximal outer tube 824, and a proximal inner drive tube 828 rotatably mounted within the proximal inner articulation tube 826. The proximal inner drive tube 828 includes a crown gear 830 at its distal end. The crown gear 830 is configured to mesh with the first spur gear 820 of the joint mechanism 812. With this arrangement, the proximal inner drive tube 828 may be driven by a motor (not shown) located at the proximal end of device 800, as with previously described embodiments. The proximal inner drive tube 828 then drives the first spur gear 820, which in turn drives the distal inner drive tube 816 in an opposite direction from that of the proximal inner drive tube 828. The distal inner drive tube 816 then rotatably drives the tissue cutter assembly 804 as previously described.

The spindle 822 pivotably interconnects the proximal end of the distal outer tube 814 with the distal end of the proximal outer tube 824, allowing the two outer tubes 814 and 824 to pivot with respect to one another. The proximal and distal inner drive tubes 828 and 816 and the first spur gear 820 are arranged such that they are able to continually drive the tissue cutter assembly 804 regardless of the orientation the distal outer tube 814 relative to the proximal outer tube 824. A gear segment 832 is provided at the proximal end of the distal outer tube 814. The proximal inner articulation tube 826 includes a crown gear 834 at its distal end that is configured to mesh with the gear segment 832 of the distal outer tube 814. Rotating the proximal end (not shown) of the proximal inner articulation tube 826, such as with a knob or other control, causes the crown gear 834 at the distal end of the proximal inner articulation tube 826 to pivot the distal portion 810 of the elongate member 806 relative to the proximal portion 808. FIG. 8B shows the distal portion 810 of the elongate member 806 in a first articulated position, shown with solid lines, and in a second articulated position, shown with phantom lines. The articulation capabilities of the joint mechanism 812 allow device 800 to approach difficult to reach target tissues from different angles.

The joint mechanism 812 may be provided with a flexible sheath, bellows or other covering (not shown) over the joint to prevent the mechanism from damaging adjacent tissue and to seal irrigation fluid that may be flowing distally and/or proximally through the joint 812. In some embodiments, irrigation fluid is provided externally adjacent to the tissue cutter assembly 804. Suction is provided at the proximal end of the proximal inner drive tube 828 to draw the irrigation fluid through the tissue cutter assembly 804 and up through the distal and proximal inner drive tubes 816 and 828, thereby transporting cut tissue debris proximally through the elongate member 806. In other embodiments, irrigation fluid may be provided distally through channels and/or tubing through the elongate member 806. In still other embodiments, irrigation fluid may be provided distally through the center of the proximal and distal inner drive tubes 828 and 816.

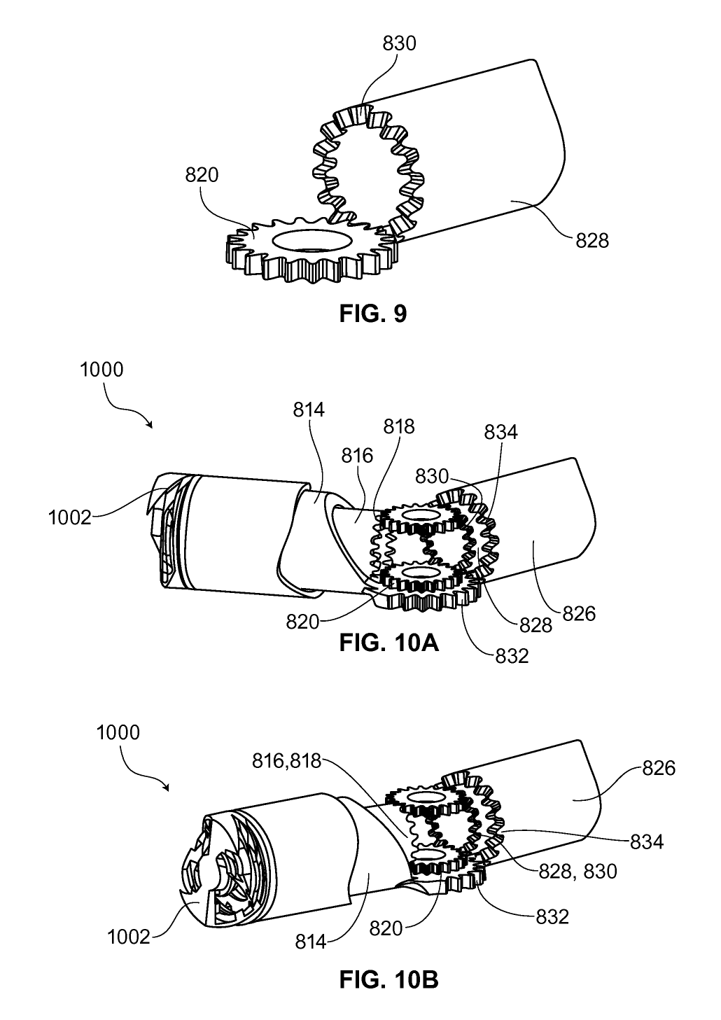

FIG. 9 is an enlarged view of the crown gear 830 at the distal end of the proximal inner drive tube 828 intermeshing with the first spur gear 820.

FIGS. 10A and 10B are enlarged fragmentary views showing a tissue debrider 1000. Device 1000 is similar to the previously described device 800 but utilizes a concentric end cutter 1002 rather than the tissue cutting assembly 804 shown in FIGS. 8A and 8B. The proximal end of the distal outer tube, the proximal outer tube, and the interconnecting spindle are not shown in FIGS. 10A and 10B for clarity. FIG. 10A shows device 1000 in an articulated orientation, and FIG. 10B shows device 1000 in a straight orientation.

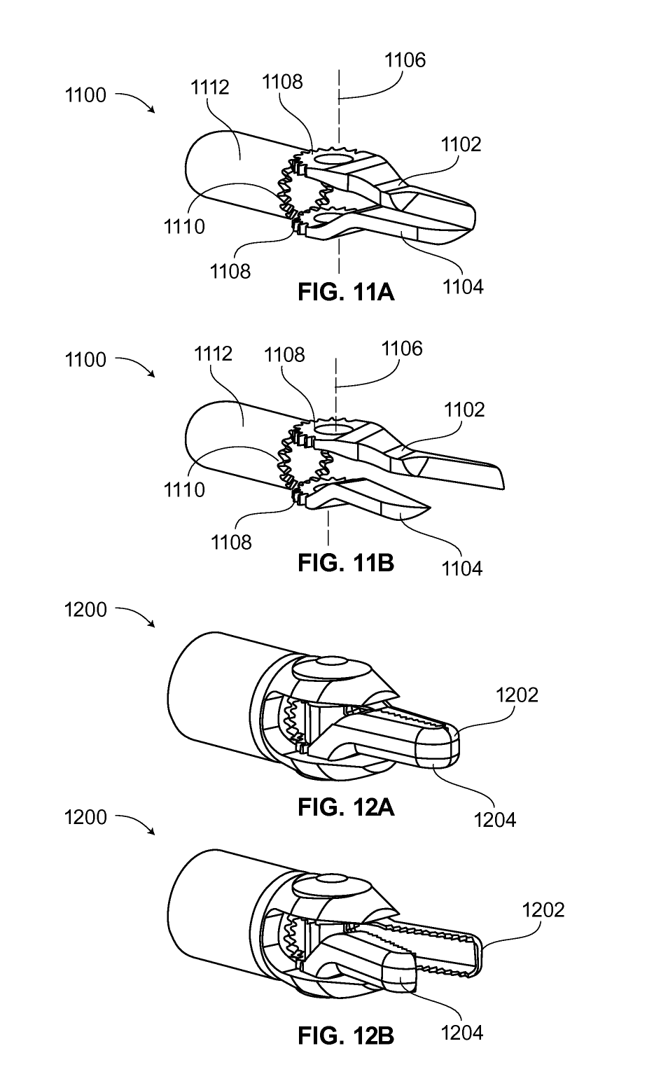

Referring to FIGS. 11A and 11B, a tissue cutting device 1100 is shown. Device 1100 includes a first tissue shearing member 1102 and a second tissue shearing member 1104 that each pivot about a common axis 1106. Each of the tissue shearing members has a gear segment 1108 located at its proximal end. The gear segments 1108 engage with a common crown gear 1110 located at the distal end of an inner drive tube 1112. As can be seen, the gear segment 1108 of the first tissue shearing member 1102 engages with the top of the crown gear 1110, while the gear segment 1108 of the second tissue shearing member 1104 engages with the bottom of the crown gear 1110. With this arrangement, turning the inner drive tube 1112 will cause the first and second tissue shearing members 1102 and 1104 to pivot in opposite directions. FIG. 11B shows the first and second tissue shearing members 1102 and 1104 in an open position. When in this position and placed over target tissue, and then pivoted in opposite directions to a closed position by turning the inner drive tube 1112 as shown in FIG. 11A, tissue is sheared between the distal cutting surfaces of the first and second tissue shearing members 1102 and 1104.

The actuation of the above tissue cutting device or scissors 1100 may be performed with high speed oscillation, such as by using a servo. By alternately driving the motor clockwise and counter-clockwise for short durations of less 500 milliseconds, a high speed oscillating scissors actuator can be achieved.

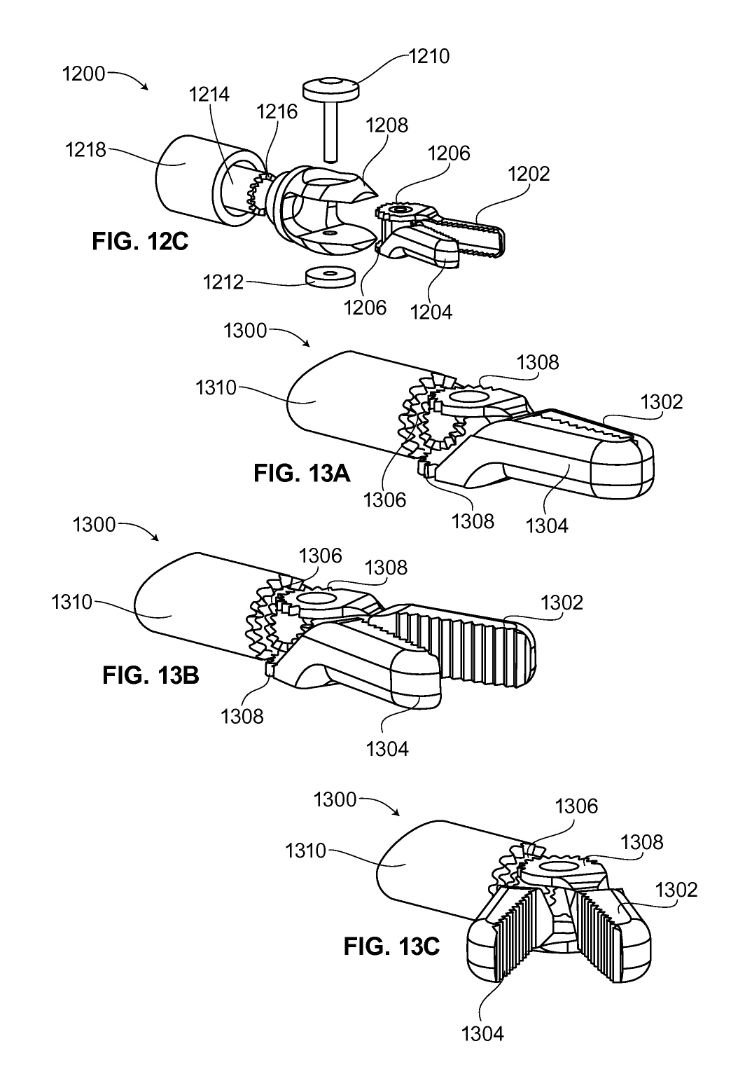

Referring to FIGS. 12A-12C, a tissue grasping device 1200 is shown. Tissue grasping device 1200 is constructed in a similar manner to that of a tissue cutting device 1100, but has opposing flat faced jaws 1202 and 1204 for grasping tissue as opposed to tissue shearing members for shearing tissue. FIG. 12A shows the jaws 1202 and 1204 in a closed position. FIG. 12 B shows the jaws 1202 and 1204 pivoted into an open position. FIG. 12 C is an exploded view showing the components of device 1200, which include: a first jaw 1202 having a first gear segment 1206, a second jaw 1204 having a second gear segment 1206, a lug or distal housing 1208, a spindle 1210 and securing washer 1212 for pivotably retaining the first and the second jaws 1202 and 1204 in the distal housing 1208, a distal inner drive tube 1214 having a crown gear 1216 at the distal end thereof for engaging with the gear segments 1206, 1206 of the first and second jaws 1202 and 1204, and a distal outer tube 1218. Similar to the drive trains of the previously described embodiments, rotating the distal inner drive tube 1214 in one direction causes the jaws 1202 and 1204 to open, and rotating the drive tube 1214 in the opposite direction causes the jaws 1202 and 1204 to close.

Referring to FIGS. 13A-13I, another embodiment of a tissue grasping device 1300 is shown. Device 1300 is constructed and operates in a manner similar to that of device 1200, but has independently driven jaws 1302 and 1304 instead of jaws that pivot open or closed together. A first inner drive tube 1306 engages a first gear segment 1308 on a first jaw member 1302 as shown. Similarly, a second inner drive tube 1310 engages a second gear segment 1308 on a second jaw member 1304 as also shown. With this arrangement, when both the first and the second inner drive tubes 1306 and 1310 are rotated in one direction, the first and second jaws 1302 and 1304 move to a closed position as shown in FIGS. 13 A and 13 D. When both the first and the second inner drive tubes 1306 and 1310 are rotated in an opposite direction, the first and second jaws 1302 one 1304 move to a open position as shown in FIGS. 13 B and 13 E. The open and closed positions can also be obtained by holding one inner drive tube and jaw member fixed while the other inner drive tube and jaw member are moved. Additionally, by rotating the first and the second inner drive tubes 1306 and 1310 in opposite directions from one another, both jaw members 1302 and 1304 can be pivoted in the same direction. For example, FIGS. 13 C and 13 F show the jaw members 1302 and 1304 in an open position but moved to one side of the central axis of the first and second inner drive tubes 1306 and 1310. With this arrangement, an infinite number of jaw movements can be obtained by driving the first and the second inner drive tubes 1306 and 1310 independently in various directions, at various speeds and time periods. Such jaw movements can be controlled manually, with computer assistance, or under complete computer control. FIG. 13 G shows a partial exploded view of major components of device 1300. FIG. 13 H is an enlarged perspective view of device 1300, including a distal housing or lug 1312, a spindle 1314, and a retaining washer 1316. FIG. 13 I is an exploded view of exemplary device 1300.

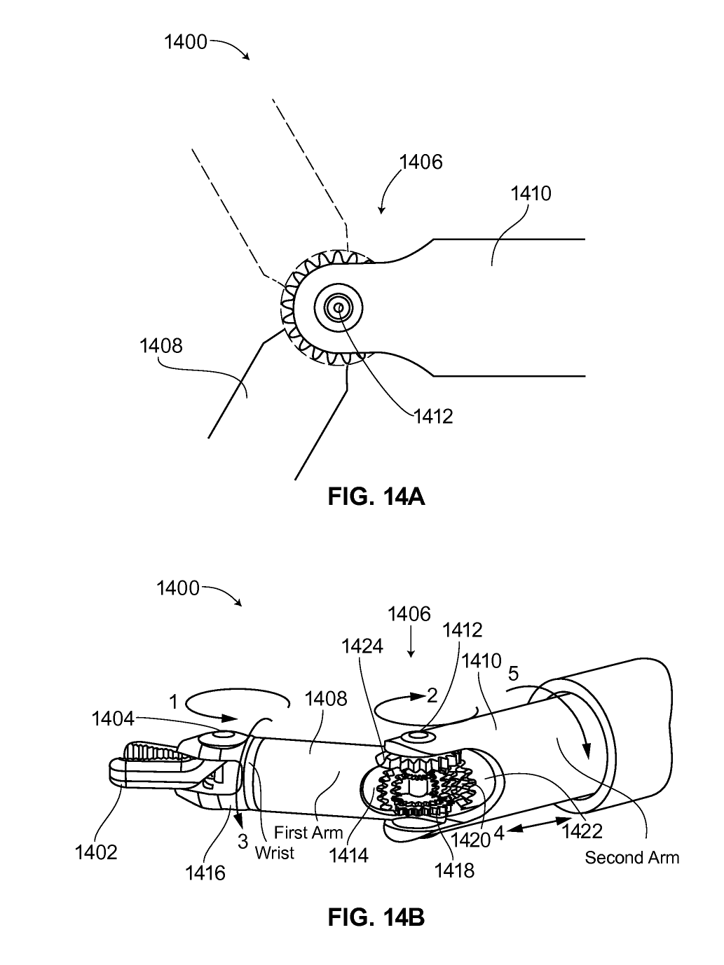

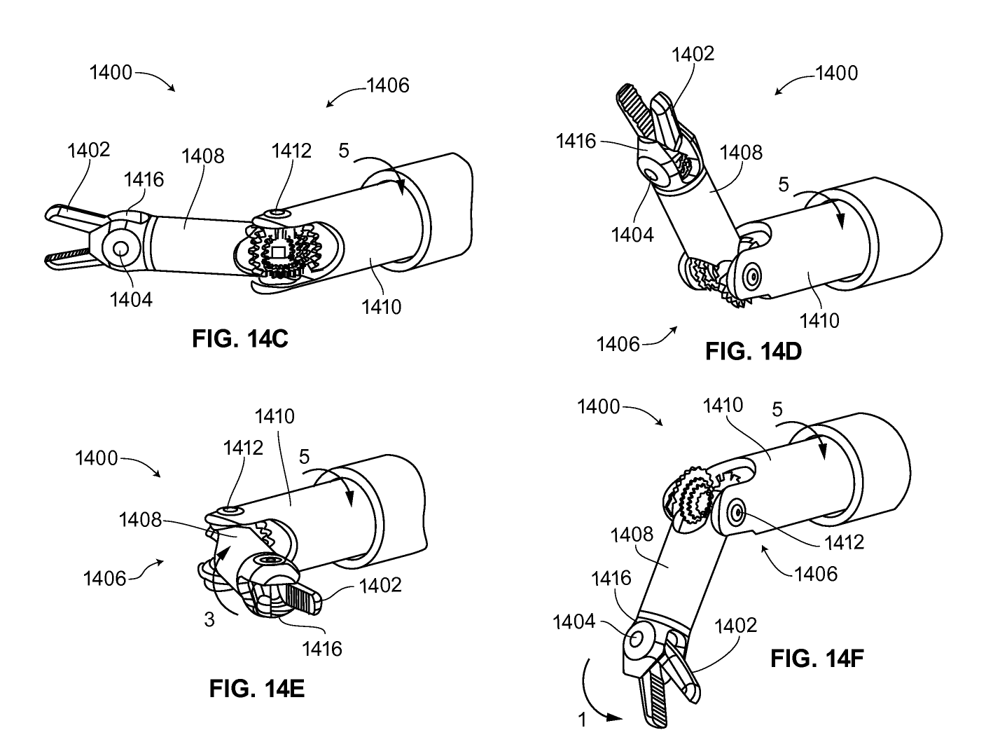

Referring to FIGS. 14 A-14 C, another exemplary tissue manipulating device 1400 having additional degrees of articulation is shown. As best seen in FIG. 14 B, the distal end of device 1400 is equipped with a tissue grasper 1402 similar to that of previously described device 1300. In other words, the first and second jaw members of the tissue grasper are independently pivotable about the spindle 1404, as shown by Arrow 1. Device 1400 is also equipped with a joint mechanism 1406 similar to that of previously described device 800. As previously indicated, the joint mechanism 1406 permits the distal portion 1408 of the elongate member to be pivoted relative to the proximal portion 1410 of the elongate member. FIG. 14 A shows a portion of device 1400, with the distal portion 1408 of the elongate member articulated about the spindle 1412 to a first position, shown in solid lines, and articulated about the spindle 1412 to a second position, shown with phantom lines.

As shown by Arrow 3 in FIG. 14B, the tissue grasper or end effector 1402 of device 1400 may also be rotated about a wrist axis. This may be accomplished by providing a third distal inner drive tube 1414 nested within the distal outer tube of the distal portion 1408 of the elongated member with the other inner drive tubes. The distal housing 1416 and the third distal inner drive tube 1414, which are rigidly coupled together, are configured to pivot relative to the distal outer tube. At least a third spur gear 1418 and a third proximal inner drive tube 1420 within the proximal portion 1410 of the elongate member are also provided for driving the distal housing 1416 about the wrist axis in a similar fashion to the operation of the other inner drive tubes. In this embodiment the proximal portion 1410 of the elongate member includes at least four inner drive tubes. The three innermost drive tubes of the proximal portion 1410 of the elongate member correspond with and drive the three innermost drive tubes of the distal portion 1408 of the elongate member through separate spur gears. More specifically, the innermost drive tubes drive the first jaw member, as shown by Arrow 1. The second innermost drive tubes drive the second jaw member, as also shown by Arrow 1. The third innermost drive tubes drive the tissue grasper assembly about the wrist axis, shown by Arrow 3. The fourth innermost drive tube 1422, found only in the proximal portion 1410 of the elongate member, engages with a gear segment 1424 on the outer tube of the distal portion 1408 of the elongated member to pivot the distal portion about the spindle axis 1412, as shown by Arrow 2.

The proximal portion 1410 of the elongate member, and the distal portion 1408 along with it, may also be driven axially inward and outward, as shown by Arrow 4. Additionally, the proximal portion 1410 of the elongate member, and the distal portion 1408 along with it, may also be rotated about its central axis, as shown by Arrow 5. Thus, device 1400 may be articulated and/or translated about five axes, as shown in FIG. 14B.

FIGS. 14 C-14 F depict various movements that can be made by device 1400. In each of these four figures, the proximal portion 1410 of the elongate member, and the distal portion 1408 along with it, is rotated 90° about the central axis of the proximal portion 1410 of the elongate member. FIG. 14E also shows the distal end effector/grasper 1402 rotated about the wrist axis, as shown by Arrow 3. Additionally, FIG. 14 F shows both the first and the second jaw members rotated about the distal spindle 1404, as shown by Arrow 1. These figures depict only a few of the many positions that can be achieved by manipulating the five axes of device 1400.

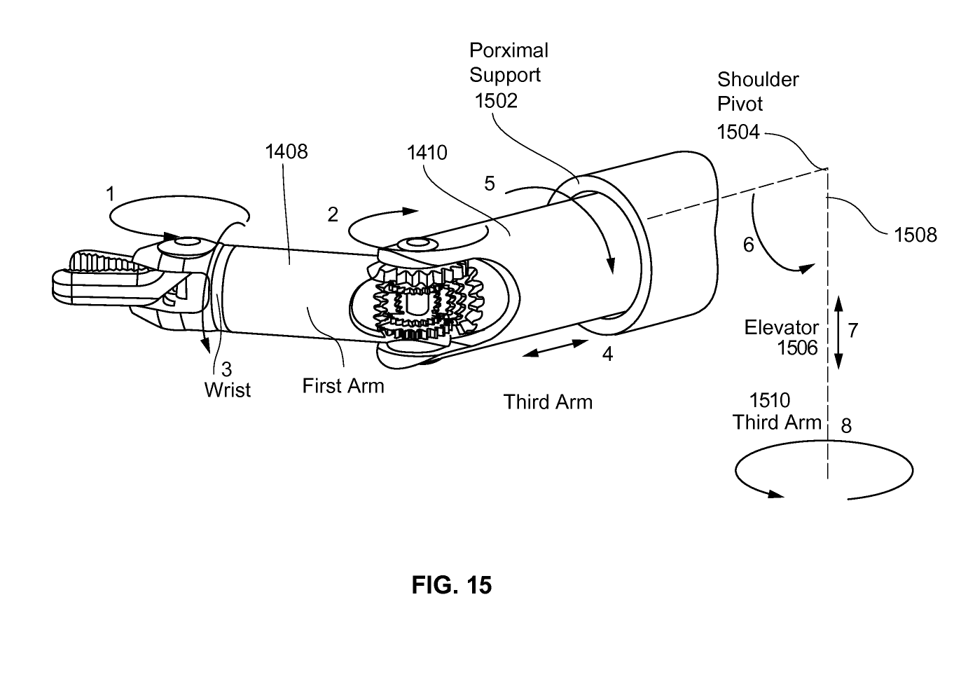

Referring to FIG. 15, an additional exemplary articulating device 1500 is shown. Everything in the distal direction from the proximal support 1502 of device 1500 may be configured the same as in previously described device 1400. Articulating device 1500 is provided with three additional degrees of freedom. More specifically, the proximal support 1502 of device 1500, and the proximal 1410 and distal portions 1408 of the elongate member along with it, may be pivoted about a shoulder joint 1504, as depicted by Arrow 6. Additionally, device 1500 may be provided with an elevator 1506 to translate the proximal support 1502 up-and-down along a vertical axis 1508, as depicted by Arrow 7. Furthermore, the proximal support 1502, supported by a third arm 1510, may be rotated about the vertical axis 1508, as depicted by Arrow 8.

Miniature robotic manipulators may be constructed using the above technology. In some embodiments, the manipulators may be configured to be set up by a surgeon and actuated to run autonomously or semi-autonomously. For example, the robotic manipulator can be configured to take a first pass at tissue removal using closed loop feedback such as torque and force sensing. A second, more delicate pass of tissue removal can then be performed by the surgeon to finish the procedure. With the first pass not taking much effort from the surgeon, surgeon fatigue can be kept to a minimum. In some embodiments, the instrument movements provided by the surgeon can be enhanced by robotic control. For example, instead of manipulating the surgical instrument directly, the surgeon can operate controls that have be configured to simulate the proximal end of the instrument. These controls in turn provide input to a computer control system that then provides outputs to prime movers such as stepper motors for driving the surgical instrument. The surgeon's movements can be modified by the computer control, such as by smoothing out the movements and/or limiting a depth of tissue cutting. Haptic feedback from the instrument can be fed back to the surgeon to more closely simulate direct control.

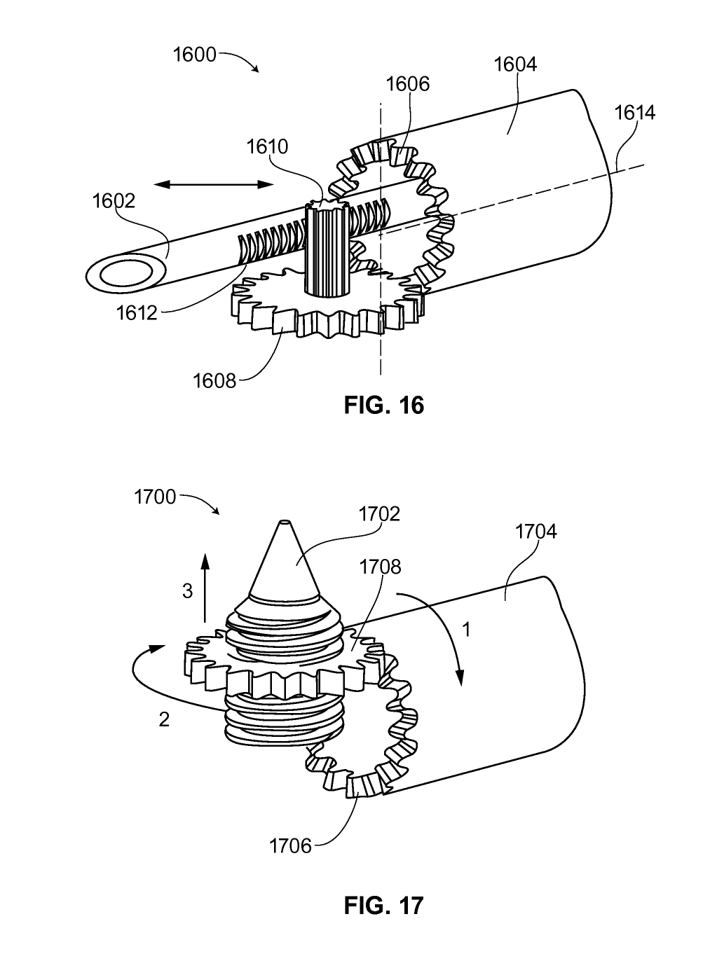

Referring to FIG. 16, an exemplary axial linear tool 1600 is shown. Tool 1600 includes a needle or piston 1602 that is driven axially in and/or out along a longitudinal axis, such as for drug delivery or fluid sampling. An inner drive tube 1604 is provided with a crown gear 1606 located at its distal end that meshes with a right angle spur gear 1608. A pinion gear 1610 is rigidly attached to the spur gear 1608. The pinion gear 1610 is configured to engage a rack of teeth 1612 located along the needle 1602. When the inner drive tube 1604 is rotated about a horizontal central axis 1614, the spur gear 1608 and the pinion gear 1610 along with it are rotated about a vertical axis. This rotation causes the needle 1602 to be driven linearly in one direction, and the opposite rotation causes the needle 1602 to be driven linearly in an opposite direction.

Referring to FIG. 17, an exemplary radial linear tool 1700 is shown. Tool 1700 includes a needle 1702, electrode, or other device that may be radially driven inward and/or outward. An inner drive tube 1704 is provided with a crown gear 1706 located at its distal end that meshes with a right angle spur gear 1708. The spur gear 1708 has a threaded central opening for receiving the radially mounted tool 7002. The radially mounted tool 1702 is threaded but includes a keyway (not shown) to prevent it from rotating. As the inner drive tube 7004 is rotated about its central axis (Arrow 1), the crown gear 1706 at its distal end causes the spur gear 1708 to rotate about a radial axis (Arrow 2). The rotation of the spur gear 1708 causes the threaded tool 1702 to translate in an outward radial direction (Arrow 3), perpendicular to the central axis. Rotation of the inner drive tube 1704 in the opposite direction causes the threaded tool 1702 to translate in an inward radial direction.

In many of the above-described surgical instruments, actuation is controlled via a crown gear driving one or more right angle gears, such as for steering a portion of the instrument off at an angle from the central axis. In combination with or separately from the steering, a crown gear arrangement can also be used to actuate tools such as graspers, scissors, debriders, and other end defectors. In some embodiments, the articulating joints of these tools have a diameter of 20 mm or less. In some embodiments, the articulating joints have a diameter of about 10 mm or about 5 mm. In other embodiments, the instruments can enable micro-invasive tools of down to 1 mm. Exemplary tools that may be constructed with this inventive technology include probes, sensors (e.g. temperature, pressure, torque, tissue impedance, infrared, radiofrequency coils, heart rate, ultrasound), staplers, tissue approximation devices, suture devices, cameras, optics, neuro-stimulation devices, ablation devices, drug delivery devices, and/or biopsy devices.

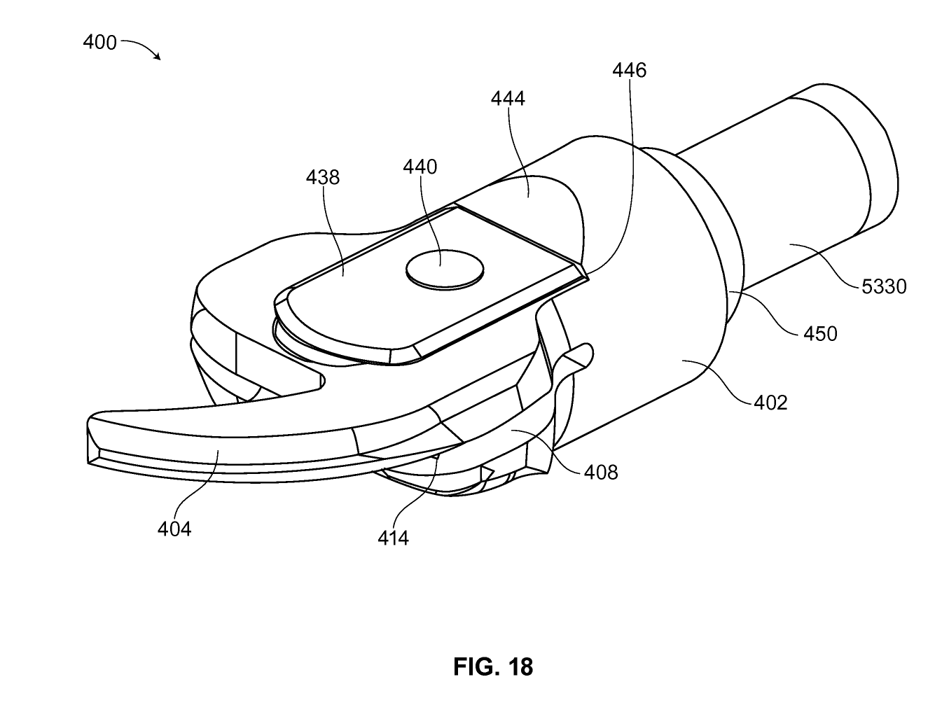

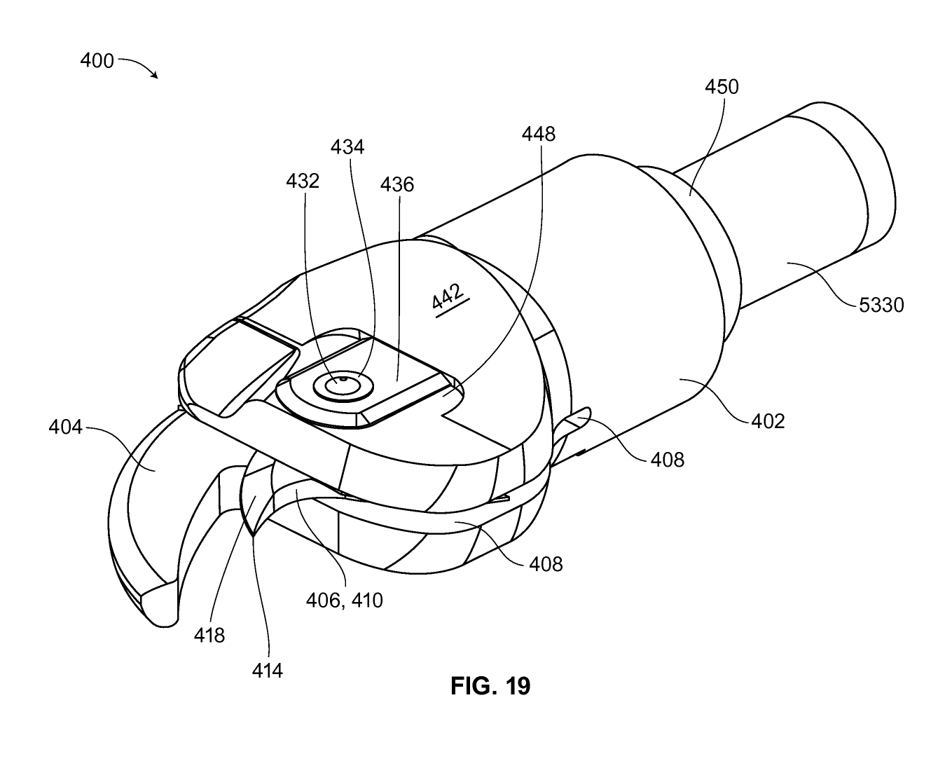

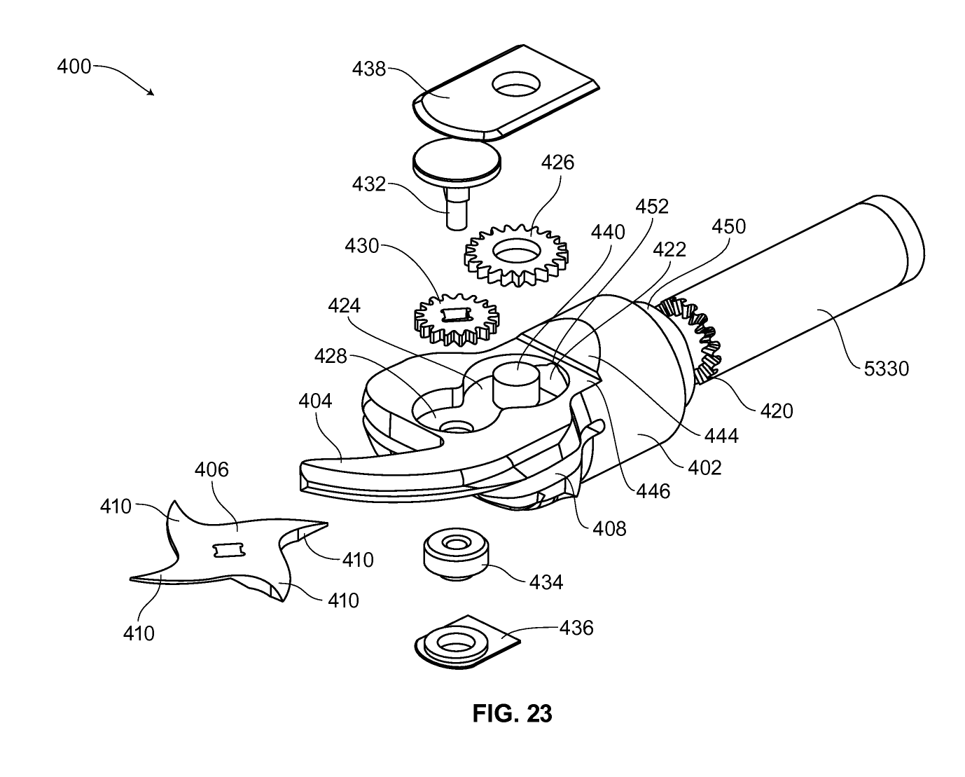

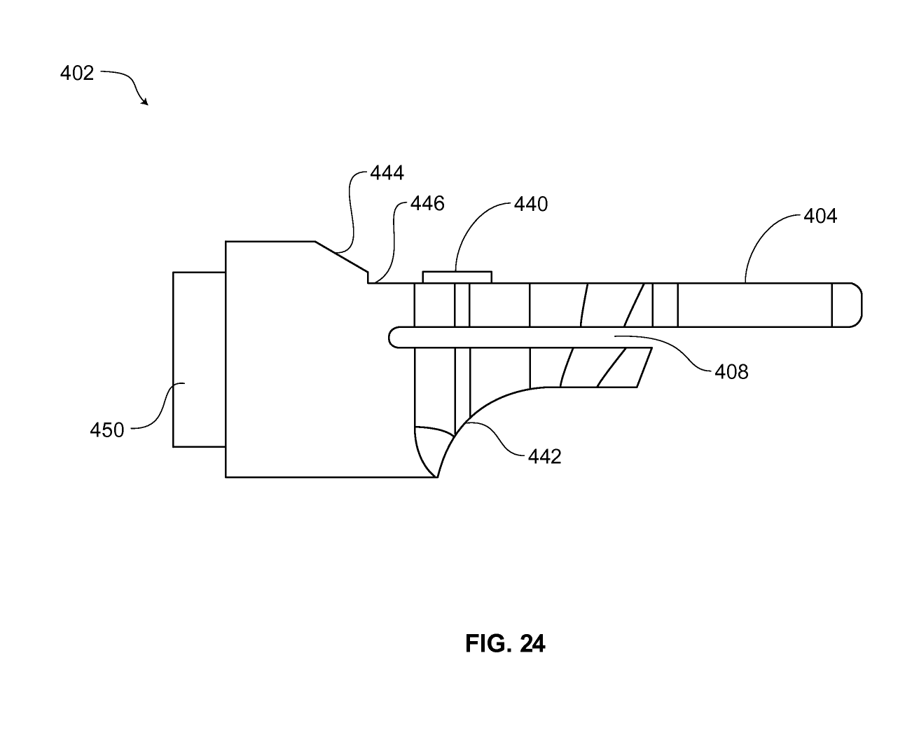

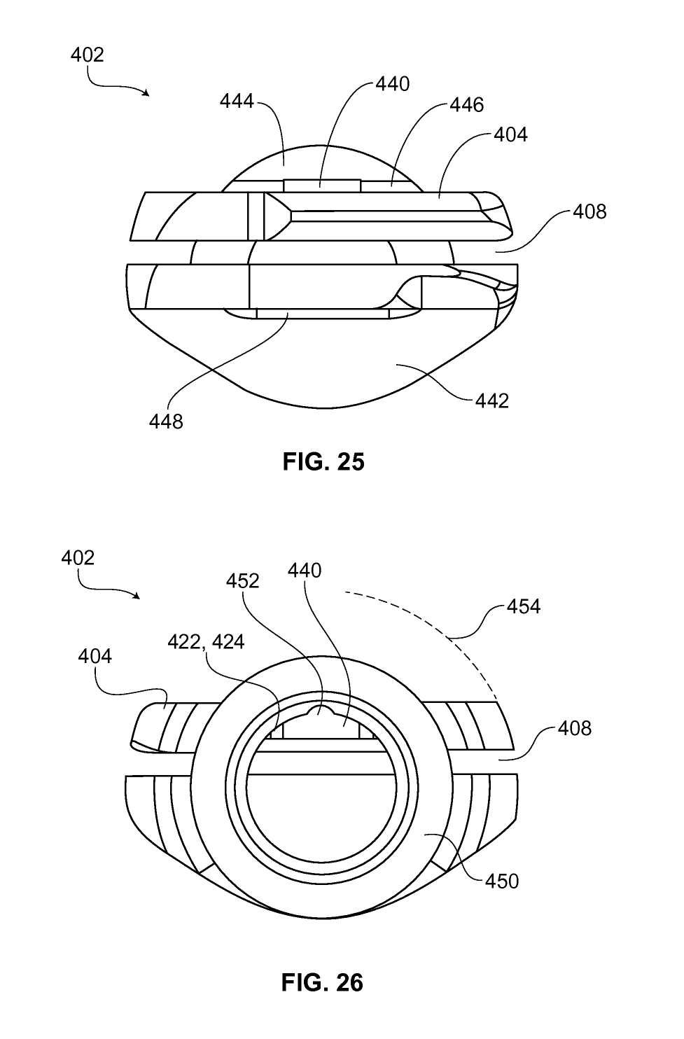

FIGS. 18-26 show another exemplary embodiment of a tissue manipulating device 400. Device 400 is a powered scissors construct that may be coupled to the distal end of any of the fixed or articulating shafts disclosed herein, or to a similar elongate member configured to introduce the device to a target tissue site of a subject. FIGS. 18 and 19 are top and bottom perspective views, respectively, showing the overall construction of device 400. As shown in these figures, device 400 includes a distal housing or lug 402 provided with a distally extending, arcuate, fixed arm 404. Rotating blade 406 is rotatably mounted within slot 408 that traverses the distal end of lug 402, as best seen in FIG. 24. Blade 406 is provided with four arcuate cutting elements 410 (as best seen in FIG. 23) that capture and shear tissue in turn between each cutting element 410 and fixed arm 404 as blade 406 rotates in the direction shown by Arrow 412. Rotating blade 406 is driven by inner drive tube 5330, as will subsequently be described in detail.

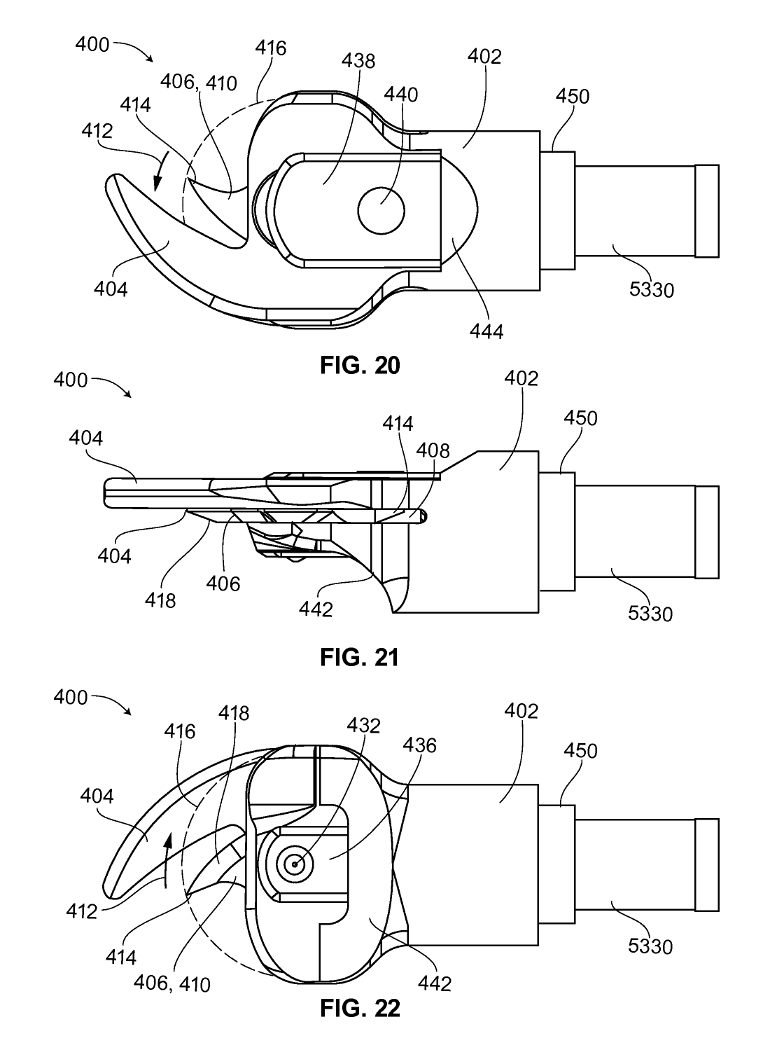

Referring to FIGS. 20-22, top, side and bottom views, respectively, are provided showing device 400 of FIGS. 18 and 19. As can be seen in these drawings, cutting elements 410 of rotating blade 406 are shorter than fixed arm 404. The outer tips 414 of cutting elements 410 travel along circular path 416 depicted by dotted lines in FIGS. 20 and 22. Cutting elements 410 are shielded from adjacent tissue during the majority of their travel around their axis of rotation by the portions of lug 402 above and below slot 408. As best seen in FIGS. 20 and 22, tissue may be cut by device 400 when it enters the space between a cutting element 410 and fixed arm 404, and is then sheared between the two elements as cutting element 410 rotates under fixed arm 404. In this exemplary embodiment, cutting elements 410 are flat on their top side, as shown in FIG. 20, and have a cutting bevel 418 provided along the bottom side of the leading edge, as shown in FIG. 22. The cutting edge of cutting element 410 is curved in the same direction as the cutting edge of fixed arm 404, namely in an outward direction trailing away from the direction of rotation. The cutting edge of cutting element 410 is provided at a slightly tighter radius than that of fixed arm 404 such that the tissue is progressively cut starting at the proximal ends of the cutting edges and moving towards the distal tip 414 of cutting element 410. In this exemplary embodiment, four cutting elements 410 are provided on blade 406, however in other embodiments more or fewer cutting elements may be provided.

Referring to FIG. 23, the drive train components of device 400 are shown. As with previously described embodiments, the distal end of inner drive tube 5330 is provided with a crown gear 420. When device 400 is assembled, a top portion of crown gear 420 is accessible through opening 422 in lug 402. An annular recess 424 is provided in the top of lug 402 for rotatably receiving a first spur gear 426. Annular recess 424 communicates with opening 422 such that first spur gear 426 can mesh with crown gear 420. Another recess 428 is provided in the top of lug 402 for rotatably receiving a second spur gear 430. When device 400 is assembled, crown gear 420 drives first spur gear 426, which in turn drives second spur gear 430. Spur gears 426 and 430 rotate about parallel axes that are each perpendicular to the central axis of rotation of crown gear 420.

Second spur gear 430 is provided with a square aperture therethrough for receiving drive pin 432. Similarly, blade 406 is provided with a square aperture therethrough. Drive pin 432 passes through second spur gear 430 and blade 406, and its distal end is received within aligner bushing 434. Aligner bushing 434 is received within a circular recess (not shown) in the bottom of lug 402. Drive pin 432 and aligner bushing 434 cooperate to rotatably mount blade 406 in a proper alignment so that it may be driven by second spur gear 430. Lower retainer cap 436 may be provided to captivate aligner bushing 434 within lug 402. Retainer cap 436 may be welded in place on the bottom of lug 402, as shown in FIG. 22. Similarly, upper retainer cap 438 may be welded in place on the top of lug 402 to rotatably captivate drive pin 432 and first and second spur gears 426 and 430 within their respective recesses in lug 402. Upper retainer cap 438 may be provided with a through hole, as best seen in FIG. 23, for engaging with the gear mounting post 440 in the center of annular recess 424.

Referring to FIGS. 24-26, further details of lug 402 are shown. Curved portion 442 may be provided along the bottom of lug 402 to aid in positioning the distal end of device 400 at the target tissue site without damaging tissue. Bevel 444 may be provided along the top of lug 402, and other features may be rounded as shown to prevent device 400 from damaging adjacent tissue. Recess 446 may be provided adjacent to bevel 444 to make a smooth transition between upper retainer cap 438 and bevel 444. Similarly, recess 448 may be provided adjacent to curved portion 442 to make a smooth transition between lower retainer cap 436 and curved portion 442. Boss 450 may be provided at the proximal end of lug 402 for engaging with the distal end of an outer shaft (not shown) of device 400. The outside diameter of lug 402 may be configured to be the same as the outside diameter of the outer shaft to create a smooth transition between the two elements. One or more fluid channels 452 may be provided along the inside diameter of lug 402, as best seen in FIG. 26, to provide cooling, lubrication and or irrigation fluid to the distal end of device 400. As shown, a fluid channel 452 may be aligned with opening 422 in lug 402 for providing fluid directly to spur gears 426 and 430 and to drive pin 432.

In some embodiments, the distal end of device 400 is configured to fit through a 10 mm trocar, endoscope or catheter, as partially depicted by dotted line 454 in FIG. 26. In other embodiments, device 400 is configured to fit through a 5 mm or smaller opening 454.

As shown and described, rotatable blade 406 of exemplary device 400 rotates about an axis that is perpendicular to an axis of rotation of inner drive tube 5330. In other embodiments (not shown), lug 402, crown gear 420 and first spur gear 426 may be configured such that the axis of rotation of rotatable blade 406 is oriented at a different angle with respect to inner drive tube 5330. In some embodiments, the angle between the two axes is 45 degrees. In other embodiments, the two axes are parallel, with the spur gear(s) located outside of the distal tip of the inner drive tube. In some embodiments, the first spur gear may be tilted downward/inward, such that its axis of rotation falls inside the inner drive tube.

As with previously described embodiments, the exemplary device 400 shown in FIGS. 23-26 can be configured to be operated manually, operated under semi-robotic control wherein the surgeon is assisted by computer in tissue cutting procedures, and or with fully robotic control wherein the tissue cutting procedures are performed automatically.

In any of the embodiments disclosed herein, the tissue manipulating device may include one or more radio frequency (RF) electrodes on the end effector. For example, tissue grasping device 1300 shown in FIGS. 13A-13I may have an electrode located on the distal housing or lug 1312, or the entire lug may form an electrode. Additionally or alternatively, first pivoting jaw member 1302 and/or second pivoting jaw member 1304 may form an electrode and/or have one or more electrodes located on it. Such electrodes may be used in a monopolar or bipolar configurations, such as for cutting, sealing, coagulating, desiccating, and/or fulgurating tissue.

In one exemplary embodiment, first pivoting jaw member 1302 forms a first RF electrode and second pivoting jaw member 1304 forms a second RF electrode of opposite polarity. In this embodiment, jaw members 1302 and 1304 are electrically insulated from each other and may also be insulated from the rest of grasping device 1300. RF energy may be provided to jaw members 1302 and 1304 by inner drive tubes 1310 and 1306, respectively, which may also be insulated from each other, and through gear segments 1308. Alternately or in combination, other electrical conductors such as insulated wires may run the length of the elongated member/instrument shaft and connect to jaw members 1302 and 1304, or electrodes located thereon. An electrical connector or cable located at the proximal end of the instrument may then be connected to an RF generator. In use, when a surgeon activates the RF energy supplied to jaws 1302 and 1304, tissue grasped between the jaws is sealed, for example, by the RF energy passing between the jaws.

In another exemplary embodiment, the scissors device 1100 shown in FIGS. 11A and 11B may be provided with RF power for enhanced cutting and/or sealing of tissue. Similar to the previously described embodiments, the cutting edges of jaw members 1102 and 1104 may each be provided with at least one electrode. In some embodiments, the entire jaw members are electrified. Portions other than the cutting edges may be covered with a ceramic coating to insulate those portions from surrounding tissue. In other embodiments, a ceramic inlay or covering may be provided on the jaw members to insulate certain portions. In still other embodiments, the jaw members can be formed from ceramic. Conductive electrodes may then be inlayed along the cutting edges of the jaw members.

In another exemplary embodiment, the cutting edge of fixed arm 404 of scissors device 400 shown in FIGS. 18-26 may be provided with an RF electrode. This electrode may cut or seal tissue independently from rotating blade 406, or blade 406 may form another electrode of opposite polarity such that tissue is cut mechanically and/or with RF energy by arm 404 and blade 406.

In other embodiments (not shown), a CMOS or CCD camera, one or more scanning fibers, other optical imaging components or suitable devices may be attached to one or more pivoting members of an instrument end effector. These components may be independently aimed or steered by pivoting the end effector member with a drive tube crown gear, as previously described.

In view of the teachings herein, many further embodiments, alternatives in design and uses of the embodiments of the instant invention will be apparent to those of skill in the art. For example, it is envisioned that the locations of the inner and outer tubes may be reversed and/or the nesting order of tubes may be varied from the embodiments disclosed herein. As such, it is not intended that the invention be limited to the particular illustrative embodiments, alternatives, and uses described above but instead that it be defined by the claims presented hereafter.

What is claimed is:

1. A medical device for manipulating tissue of a subject, comprising:
a distal housing configured with an end effector;
an elongate member coupled to the distal housing and configured to introduce the distal housing to a target tissue site of the subject, the elongate member comprising a proximal portion having a first central axis and a distal portion having a second central axis, the proximal portion of the elongate member comprising a proximal outer tube and a proximal inner drive tube rotatably mounted within the proximal outer tube, the distal portion of the elongate member comprising a distal outer tube and a distal inner drive tube rotatably mounted within the distal outer tube, the distal inner drive tube engaging with a portion of the end effector to drive the end effector;
a joint mechanism configured to pivotably connect a distal end of the proximal outer tube with a proximal end of the distal outer tube, wherein the joint mechanism allows the distal portion of the elongate member to be pivoted relative to the proximal portion such that an angle formed between the first and the second central axes can be changed;
a proximal crown gear located at a distal end of the proximal inner drive tube;
a distal crown gear located at a proximal end of the distal inner drive tube; and
a first spur gear spanning between and inter-engaging with the proximal crown gear and the distal crown gear, thereby allowing the end effector to be positioned by the proximal and the distal outer tubes, and to be driven by the proximal inner drive tube, the spur gear and the distal inner drive tube.

2. The medical device of claim 1, wherein the end effector comprises a rotary tissue cutter assembly.

3. The medical device of claim 2, wherein the rotary tissue cutter assembly comprises at least one rotatable member that rotates about the second central axis.

4. The medical device of claim 2, wherein the rotary tissue cutter assembly comprises at least one rotatable member that has an axis of rotation that is perpendicular to the second central axis.

5. The medical device of claim 2, wherein the distal inner drive tube comprises a first lumen and the proximal inner drive tube comprises a second lumen, wherein the first lumen is in fluid communication with the tissue cutter assembly and the second lumen is in fluid communication with the first lumen through the joint mechanism.

6. The medical device of claim 5, wherein the tissue cutter assembly, the first lumen, the joint mechanism and the second lumen are configured to cooperate to transport tissue debris cut by the tissue cutter assembly in a proximal direction through the first lumen, the joint mechanism and the second lumen.

7. The medical device of claim 1, wherein the end effector comprises a pair of scissor blades configured to cut tissue.

8. The medical device of claim 1, wherein the end effector comprises a pair of tissue grasper jaws.

9. The medical device of claim 1, wherein the end effector comprises a needle driver.

10. The medical device of claim 1, wherein the proximal portion of the elongate member further comprises a proximal inner articulation tube rotatably mounted within the proximal outer tube, and wherein the proximal inner articulation tube includes a crown gear on a distal end thereof configured to mesh with a gear segment of the joint mechanism to pivotably drive the distal portion of the elongate member relative to the proximal portion of the elongate member.

11. The medical device of claim 1, wherein the proximal portion of the elongate member comprises a second proximal inner drive tube rotatably mounted within the proximal outer tube, wherein the distal portion of the elongate member comprises a second distal inner drive tube rotatably mounted within the distal outer tube, the second distal inner drive tube engaging with a portion of the end effector to drive the end effector, wherein the device further comprises a second proximal crown gear located at a distal end of the second proximal inner drive tube, a second distal crown gear located at a proximal end of the second distal inner drive tube, and a second spur gear spanning between and inter-engaging with the second proximal crown gear and the second distal crown gear.

12. The medical device of claim 11, wherein the end effector comprises a pair of tissue grasper jaws, wherein one of the pair of tissue grasper jaws is configured to be rotatably driven by a crown gear located on a distal end of the first distal inner drive tube, and wherein the other of the pair of tissue grasper jaws is configured to be rotatably driven by a crown gear located on a distal end of the second distal inner drive tube, such that each of the pair of tissue grasper jaws may be independently rotated relative to the second central axis and may be rotated between an open jaw position and a closed jaw position.

13. The medical device of claim 1, wherein the proximal portion of the elongate member comprises a second proximal drive tube rotatably mounted coaxially with the proximal outer tube, wherein the distal portion of the elongate member comprises a second distal drive tube rotatably mounted coaxially with the distal outer tube, the second distal drive tube engaging with a portion of the end effector to support the end effector, wherein the device further comprises a second proximal crown gear located at a distal end of the second proximal drive tube, a second distal crown gear located at a proximal end of the second distal drive tube, and a second spur gear spanning between and inter-engaging with the second proximal crown gear and the second distal crown gear, and wherein the rotational orientation of the end effector relative to the second central axis may be changed by rotating the second distal drive tube with the second proximal drive tube and second spur gear.

14. The medical device of claim 13, wherein the proximal and the distal portions of the elongate member are configured to rotate together about the first central axis relative to a more proximal portion of the device.

15. The medical device of claim 13, wherein the proximal and the distal portions of the elongate member are configured to translate together about the first central axis relative to a more proximal portion of the device.

16. The medical device of claim 13, wherein the proximal and the distal portions of the elongate member are configured to pivot together about a shoulder joint relative to a more proximal portion of the device.

17. The medical device of claim 13, wherein the proximal and the distal portions of the elongate member are configured to translate together in a direction perpendicular to the first central axis relative to a more proximal portion of the device.

18. The medical device of claim 13, wherein the proximal and the distal portions of the elongate member are configured to pivot together about an axis perpendicular to the first central axis relative to a more proximal portion of the device.

19. The medical device of claim 1, further comprising a second spur gear spanning between and inter-engaging with the proximal crown gear and the distal crown gear, thereby allowing the end effector to be driven by the proximal inner drive tube, the first and second spur gears and the distal inner drive tube, wherein the first and the second spur gears provide a dual load path between the proximal and the distal inner drive tubes.

20. A method of manipulating tissue of a subject comprising:
providing a device having a distal housing configured with an end effector and an elongate member coupled to the distal housing;
introducing the distal housing to a target tissue site of the subject with the elongate member;
driving the end effector with a drive train comprising a proximal crown gear located at a distal end of a proximal drive tube, a distal crown gear located at a proximal end of a distal drive tube, and a first spur gear spanning between and inter-engaging with the proximal crown gear and the distal crown gear;
pivoting the location of the end effector, the distal housing and the distal drive tube relative to the proximal drive tube by rotating a second proximal tube, the second proximal tube being rotatably mounted coaxially with the proximal drive tube and having a crown gear located on a distal end, the crown gear engaging with a gear segment coaxially mounted with the spur gear; and
manipulating the tissue of the subject with the end effector.

21. The method of claim 20, wherein the end effector comprises a rotary tissue cutter assembly.

22. The method of claim 21, wherein the rotary tissue cutter assembly comprises at least one rotatable member that rotates about a central axis of the distal drive tube.

23. The method of claim 21, wherein the rotary tissue cutter assembly comprises at least one rotatable member that has an axis of rotation that is perpendicular to a central axis of the distal drive tube.

24. The method of claim 20, wherein the end effector comprises a pair of scissor blades configured to cut tissue.

25. The method of claim 20, wherein the end effector comprises a pair of tissue grasper jaws.

26. The method of claim 20, wherein the end effector comprises a needle driver.

27. The method of claim 20, wherein the pivoting step comprises a computer receiving movement inputs from a surgeon and providing electrical outputs to drive an electric motor coupled to the second proximal tube.

28. A powered scissors device comprising:
a distal housing having a fixed cutting arm located thereon;
an elongate member coupled to the distal housing and configured to introduce the distal housing to a target tissue site of the subject, the elongate member comprising an outer tube and an inner drive tube rotatably mounted within the outer tube;
a rotatable blade rotatably mounted to the distal housing, the rotatable blade having at least one cutting element configured to cooperate with the fixed arm to shear tissue therebetween;
a crown gear located at a distal end of the inner drive tube; and
a first spur gear configured to inter-engage with the crown gear and coupled with the rotatable blade to allow the crown gear to drive the rotatable blade.

29. The method of claim 28, wherein the rotatable blade has an axis of rotation that is perpendicular to an axis of rotation of the inner drive tube.

30. The method of claim 28, wherein the rotatable blade is partially located within a slot formed within the distal housing such that the at least one cutting element is covered by the distal housing during at least half of its rotation about an axis of rotation of the rotatable blade.

31. A medical device for manipulating tissue of a subject, comprising:
a distal housing configured with an end effector, the end effector comprising a first member pivotably mounted to the distal housing and a second member pivotably mounted to the distal housing independent from the first member; the first and the second members each having surfaces configured to manipulate tissue of the subject; and
an elongate member coupled to the distal housing and configured to introduce the distal housing to a target tissue site of the subject, the elongate member comprising a first drive tube and a second drive tube coaxially mounted within the first drive tube, the first and the second drive tubes being configured to independently rotate relative to the distal housing, the first drive tube having a first crown gear located on a distal end thereof coupled with the first member such that rotating the first drive tube and first crown gear causes the first member to pivot, the second drive tube having a second crown gear located on a distal end thereof coupled with the second member such that rotating the second drive tube and second crown gear causes the second member to pivot,
wherein the tissue engaging surfaces of the first and the second members may be alternately pivoted towards each other by their respective drive tubes into a closed position and away from each other into an open position.

32. The medical device of claim 31, wherein the first and the second members may be pivoted in the same direction by their respective drive tubes such that an articulation angle of the members relative to the distal housing when in the closed position may be varied.

33. The medical device of claim 31, wherein the first member and the second member both pivot about a common axis.

34. The medical device of claim 31, wherein at least one of the first and the second members pivots about an axis that is transverse to an axis of rotation of the first and the second drive tubes.

35. The medical device of claim 31, wherein the first and the second members form tissue graspers.

36. The medical device of claim 31, wherein the first and the second members form tissue scissors.

37. The medical device of claim 31, further comprising a first gear segment coupled to the first member and configured to mesh with the first crown gear for pivotably driving the first member, and a second gear segment coupled to the second member and configured to mesh with the second crown gear for pivotably driving the second member.

38. The medical device of claim 37, wherein the first and the second gear segments are located on opposite sides of a central rotation axis of the first and the second drive tubes such that the drive tubes are rotated in a common direction to drive the first and the second members from the open position to the closed position.

39. The medical device of claim 31, further comprising at least one radio frequency electrode located on one of the tissue manipulating surfaces of the first and the second members.

40. The medical device of claim 31, further comprising a third drive tube configured to rotate the end effector relative to the elongate member.
